(19) United States
(12) Patent Application Publication
(10) Pub. No.: US 2014/0065688 A1
(43) Pub. Date: Mar. 6, 2014

(54) METHODS AND COMPOSITIONS FOR MAGNETOPHORETIC SEPARATION OF BIOLOGICAL MATERIALS

(71) Applicants: Shashi K. Murthy, Newton, MA (US); Laura H. Lewis, Boston, MA (US); Brian D. Plouffe, Smithfield, RI (US)

(72) Inventors: Shashi K. Murthy, Newton, MA (US); Laura H. Lewis, Boston, MA (US); Brian D. Plouffe, Smithfield, RI (US)

(73) Assignee: NORTHEASTERN UNIVERSITY, Boston, MA (US)

(21) Appl. No.: 13/983,270

(22) PCT Filed: Feb. 3, 2012
(86) PCT No.: PCT/US2012/023864

§ 371 Date: Nov. 12, 2013

Related U.S. Application Data
(60) Provisional application No. 61/439,170, filed on Feb. 3, 2011.

(51) Int. Cl. B03C 1/015; H05K 3/10; C12N 13/00

(52) U.S. Cl. CPC B03C 1/015; C12N 13/00; H05K 3/103
USPC 435/173.9; 422/527; 29/850

(57) ABSTRACT

Disclosed herein are devices for the magnetophoretic separation of target biological materials including a separation chamber that has a plurality of channels, and one or more wires carrying a current, the wires generating a magnetic force that deflects magnetically-labeled target biological materials into a buffer stream. In addition, methods of separating target biological materials from non-target biological materials in a sample are disclosed. Finally, methods for constructing a magnetophoretic separation device are disclosed.

### PRIORITY CLAIM

This disclosure claims the benefit of priority of U.S. Provisional Application No. 61/439,170, filed Feb. 3, 2011, the entire disclosure of which is relied on and incorporated into this application by reference.

### FEDERALLY SPONSORED RESEARCH OR DEVELOPMENT

This invention was sponsored through grants from the IGERT Nanomedicine and Science Program (NSF-DGE-0504331) and the National Science Foundation through grant CBET-0827868 and grant 0932195. Thus, the U.S. government has certain rights in this application.

### FIELD OF THE INVENTION

The invention is generally directed to medicine and bioengineering. More specifically, the invention is directed to medical diagnostics and tissue engineering.

### BACKGROUND

The separation of a pure cell population from heterogeneous suspensions is a vital step that precedes analytical or diagnostic characterization of biological samples. The separation of key cell populations, such as circulating tumor cells and endothelial progenitor cells, can provide valuable insight into the prognosis and progression of certain diseases. Additionally, gaining this information in a minimally invasive fashion, such as through analysis of a blood sample, reduces the need for biopsies and invasive surgeries.

Cell separation techniques may be broadly classified into two categories: those based on size and density, and those based on affinity (chemical, electrical, or magnetic). Techniques that achieve separation based on size and density are generally unable to provide adequate resolution between cell populations known to be of similar size. Affinity-based approaches, such as cell adhesion chromatography and dielectrophoresis, are alternative methods to separate cell populations, but these techniques are still limited in the efficiency and purity of cell capture. Furthermore, once target cells are isolated, recovery of viable cells for further application remains a challenge. Another affinity-based technique is fluorescence activated cell sorting (FACS), where antibodies tagged with fluorescent dyes are attached to cells in mixed suspensions via receptor-ligand binding. These cells are then sorted individually based on their fluorescence and light scattering properties. Although this technique can provide highly pure (95% or higher) cell populations, it requires expensive equipment and has limited throughput (˜10⁷ cells/hour).

The technique of magnet-activated cell sorting (MACS) allows target cell separation to be carried out in parallel, providing rapid separation (˜10″ cells/hour) of high-purity cell populations. However, operation of commercially-available MACS systems requires many processing steps, including several pre-processing and washing procedures, rendering it a very time-consuming batch-wise procedure. To overcome some of these limitations, techniques based on continuous flow separation of magnetically-tagged cells have been investigated. These analytical tools are typically bulky and require large volumes of sample (>5 mL) for operation. The advancement of MACS technology over the last 5-10 years has focused on miniaturization of the continuous flow analysis chambers to the micron scale. These microscale fluidic devices, or microfluidic channels, allow for the analysis of significantly smaller sample volumes while maintaining comparable purity of target cells within the collection suspension. Nonetheless, the current state-of-the-art in microfluidic MACS technology is still limited in throughput in comparison to other continuous flow methods. Moreover, these microfluidic MACS designs are often based on Edisonian methods of device design arrived at after multiple operational iterations rather than from rational design derived from a systematic physical approach.

Accordingly, there remains a need for a robust platform for the enumeration of a target cell population with high collection efficiencies. Additionally, there remains a need to isolate pure populations with minimal biological perturbation and efficient off-chip recovery to enable sub-cellular analyses of these cells for applications in personalized medicine.

### SUMMARY

The instant disclosure describes devices for the magnetophoretic separation of target biological materials, including a separation chamber that has a plurality of channels, and one or more wires carrying a current. In some embodiments, the wires generate a magnetic force that deflects magnetically-labeled target biological materials into a buffer stream. In one aspect, the disclosed magnetophoretic separation devices comprise a separation chamber comprising a plurality of channels that provide two or more streams. The streams comprise a sample stream comprising target biological materials and non-target biological materials in which the target biological materials are magnetically-labeled and a buffer stream that is substantially free of the sample. In certain embodiments, the one or more streams combine in a single collection channel without fluidic mixing and one or more wire(s) carrying a current, the wires generating a magnetic force that deflects the one or more magnetically-labeled target biological materials into the buffer stream.

In some embodiments, the device comprises one or more wires carrying a current. In some embodiments, the device comprises one wire. In other embodiments, the device comprises two wires. In some embodiments, the two wires are in a parallel alignment with each other. In other embodiments, the devices comprise two or more wires.

In some embodiments, the separation chamber is separated from the one or more wires by a vertical distance of about 10 microns to about 500 microns.

In some embodiments, the target biological materials are cells, proteins, solutes, or particulates susceptible to a magnetic field.

In some embodiments, the target biological materials are from peripheral whole blood, tissue digestate, amniotic fluid, umbilical cord blood, fine needle aspirates, vitreous humor biopsies, cerebrospinal fluid, or other biological fluids.

In some embodiments, the cells are rare cells compared to the total number of cells in the sample. In some embodiments, the rare cells are peripheral hematopoietic stem cells, endothelial progenitor cells, circulating tumor cells, mature circulating endothelial cells, amniotic stem cells, mesenchymal stem cells, adipose-derived stem cells, intestinal stem cells, skin stem cells, neural stem cells, cancer stem cells, adult stem cells, fetal stem cells, or progenitor cells.

In another aspect, methods of separating target biological materials from non-target biological materials in a sample are disclosed. The methods comprise labeling the target biological materials in a sample with a magnetic tag and introducing the sample into at least a first inlet of at least a first channel in a magnetophoretic separation device. The methods also comprise introducing a buffer into a second inlet of a second channel of the magnetophoretic separation device, generating a magnetic force by providing a current in one or more wire(s) placed adjacent to the channels, thereby deflecting the labeled target biological materials into the channel carrying the buffer, and collecting the target biological materials from an outlet of the second channel.

In some embodiments, the one or more wire(s) carrying a current is a single wire. In other embodiments, the one or more wires are two, three, four, five, or more wires.

In some embodiments, the target biological materials used in the methods are from peripheral whole blood, tissue digestate, amniotic fluid, umbilical cord blood, fine needle aspirates, vitreous humor biopsies, cerebrospinal fluid, or other biological fluids. In some embodiments, the target biological materials are cells, proteins, solutes, or particulates susceptible to a magnetic field.

In some embodiments, the methods use cells that are rare cells compared to the total number of cells in the sample. In some embodiments, the rare cells are peripheral hematopoietic stem cells, endothelial progenitor cells, circulating tumor cells, mature circulating endothelial cells, amniotic stem cells, mesenchymal stem cells, adipose-derived stem cells, intestinal stem cells, skin stem cells, neural stem cells, cancer stem cells, adult stem cells, fetal stem cells, or progenitor cells.

In another aspect, methods of constructing a magnetophoretic separation device are disclosed. The methods comprise providing a substrate and constructing a separation chamber on the substrate, wherein the separation chamber comprises a plurality of channels, wherein one or more sample channels combine with a buffer channel in a single collection channel. The methods further comprise constructing one or more wire(s) carrying a current on the substrate adjacent to the separation chamber.

In some embodiments, the methods use one or more wire(s) carrying a current. In some embodiments, one, two, three, four, five, or more wires are used. In some embodiments, the methods further include constructing an alignment guide that aligns the separation chamber with the one or more wires. In some embodiments, the methods include using a separation chamber that is separated from the one or more wires by a vertical distance of about 10 microns to about 500 microns.

### DESCRIPTION OF THE FIGURES

The following figures are presented for the purpose of illustration only, and are not intended to be limiting.

FIGS. 1A-C are schematic illustrations of one embodiment of the disclosed magnetophoretic separation device. FIG. 1A illustrates the configuration of a single current-carrying wire as part of the magnetophoretic separation device. FIG. 1B shows a mathematical configuration of a single current-carrying wire located at (0,0) with current flowing in the positive y-direction (out of the page). FIG. 1C is a schematic illustration of the separation device displacing target cells from the sample stream into the buffer stream, while non-target cells remain in the sample stream.

FIGS. 2A-D are schematic illustrations of one embodiment of the magnetophoretic separation design using dual wires. FIG. 2A is a graphical illustration of the dual-wire magnetophoretic separation device. FIG. 2B is a cross-sectional illustration of magnetic flux lines resulting from anti-parallel dual-wire configuration driving cell-particle complexes to the middle of device. FIG. 2C shows an injected sample split into two streams that sheath a central buffer stream. FIG. 2D is a photograph of a microfluidic chip with cell and buffer inlets (left), outlet tubing to collection tubes (right), and the chip aligned on the electromagnet wire array.

FIG. 3 is a schematic illustration of a cross-section of a printed circuit board electromagnetic array along with a PDMS microfluidic device used in evaluation of Joule heating constraints using a rational device design.

FIG. 4 is a surface plot illustrating maximum displacement achievable as a function of volumetric flow rate (μL, min⁻¹) and current (A) derived in Eq. [24], discussed herein.

FIG. 5 is a surface plot of the displacement of cell-particle complex on a standard glass coverslip (60 (L)×24 (W)×0.15 (H) mm) compared with current literature values of the isolation of target cell populations. FIG. 5 illustrates that the disclosed devices can effectively meet and exceed the processing speeds or throughputs of both commercial systems (60 μL, min-1) and microfluidic devices (11.7-100 μL, min-1).

FIG. 6 is a graphical representation illustrating that the distribution in cell and particle parameters constrains the true maximum displacement achievable.

FIG. 7 is a graphical representation showing that the capture efficiency was shown to be nearly 100% for homogenous samples of the 10-10,000 Michigan Cancer Foundation-7 (MCF-7) cells injected into an embodiment of the disclosed magnetophoretic separation device.

FIGS. 8A-D are graphical representations of experimental results using embodiments of the invention to isolate target MCF-7 cells. FIGS. 8A-B depict the results of experiments conducted with a 250 μm wide microfluidic channel at a sheath and sample flow rate of 120 μL, min⁻¹ and a current of 0.25 A. FIGS. 8C-D depict the influence of current and flow rate on cell isolation.

FIG. 9 depicts a quantitative reverse transcription-polymerase chain reaction (qRT-PCR) standard curve relating total number of cells (N) to a corresponding mass of RNA (M_{RNA}) value.

FIGS. 10A-D show the behavior of cells in culture to assess the impact of the separation process on the cells and show no clear differences in displaced cells versus non-displaced cells. FIG. 10A shows that a population of 1000 MCF-7 cells per 250 μL, was plated as a comparative control. The control cells were judged against the cells depicted in FIG. 10C, which were incubated with particles but not run through the device. FIG. 10B depicts cells that were isolated using the device without particle attachment (no displacement). FIG. 10D depicts cells tagged with magnetic microbeads and displaced within the device. Scale bars represent 50 μm. Dark spots can be seen in FIGS. 10C-D, which indicate residual microparticles on the cell surface during culture and spreading.

FIG. 11 depicts surface plots for the maximum displacement for a blood-based displacement platform (Plots 4-6) compared to a buffer-based device (Plots 1-3).

FIGS. 12A-C depict bright field micrographs illustrating the channels of the disclosed separation devices that include sample and buffer streams. FIG. 12A depicts the blood-buffer stream with a side channel flow of 120 μL min⁻¹ and a center buffer stream flow rate of 160 μL min⁻¹. FIG. 12B depicts the hydrodynamic focusing of the buffer stream between the two blood streams, where the buffer stream is approximately 100 microns in width, while the blood streams are both 75 microns in width. FIG. 12C depicts the blood streams being segregated from the collection outlet, which allows for only the target cells to be isolated.

### DETAILED DESCRIPTION

The instant disclosure relates to devices for the magnetophoretic separation of cells from biological samples. In addition, methods of separating target biological materials from non-target biological materials in a sample are disclosed. Finally, methods for constructing a magnetophoretic separation device are disclosed. In some embodiments, the disclosure describes a magnet-based separation platform within a microfluidic device. The device was designed using a mathematical rational optimization approach. The disclosed devices and methods result in a magnetic force that moves biological materials that are tagged magnetically, such as with magnetic beads, into a separate stream than non-tagged biological materials. As a result, target biological materials, such as cells, are collected in a stream that is separate from the non-target biological materials.

The disclosed devices and methods can be used in tissue engineering and diagnostic medicine. The capture of a target biological material, such as a cell, from suspension allows isolation of such cells for engineering of various tissues, such as for replacement tissue grafts. Isolation of other target cells types can provide information for the fields of diagnostic medicine and personalized medicine. The disclosed devices and methods also allow isolation of target cell types from heterogeneous suspensions with minimal pre-processing steps.

All publications, patent applications, patents, and other references mentioned herein are incorporated by reference in their entirety. In case of conflict, the present specification, including definitions, will control. In addition, the materials, methods, and examples are illustrative only and not intended to be limiting.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### DEFINITIONS

For convenience, certain terms employed in the specification, examples and claims are described below. The initial definition provided for a group or term provided in this disclosure applies to that group or term throughout the present disclosure individually or as part of another group, unless otherwise indicated.

In general, the compositions of the disclosure can be alternately formulated to comprise, consist of, or consist essentially of, any appropriate components disclosed in this disclosure. The compositions of the disclosure can additionally, or alternatively, be formulated so as to be devoid, or substantially free, of any components, materials, ingredients, adjuvants or species used in the prior art compositions or that are otherwise not necessary to the achievement of the function and/or objectives of the present disclosure.

The articles “a” and “an” are used in this disclosure to refer to one or more than one (i.e., to at least one) of the grammatical object of the article. By way of example, “an element” means one element or more than one element.

The term “or” is used in this disclosure to mean, and is used interchangeably with, the term “and/or,” unless indicated otherwise.

The term “about” is used in this disclosure to mean a given numerical value plus or minus 20% of the value.

The term “rare cells” refers to cells that are low in number as compared to a total number of cells in a particular population. For example, rare cells occur in a particular population in the range of rare cell type to other cell type in the ratio of about 1:100 to about 1:10⁹. Examples of rare cells in humans include but are not limited to peripheral hematopoietic stem cells, endothelial progenitor cells, circulating tumor cells, mature circulating endothelial cells, amniotic stem cells, mesenchymal stem cells, adipose-derived stem cells, intestinal stem cells, skin stem cells, neural stem cells, cancer stem cells, adult stem cells, fetal stem cells, or progenitor cells.

### General

The disclosure provides, in part, microfluidic devices and methods that allow for magnetophoretic separation of biological materials.

Advantages of the disclosed devices and methods include fewer pre-processing and washing procedures than conventional methods of biological material separation. In some embodiments, the separation chamber and one or more wires are constructed as separate parts of the device. By constructing these pieces separately, the one or more wires can be re-used, while the separation chamber that comes in contact with biological materials, some of which is potentially biohazardous, can be disposed. This construction process has the advantage of reducing waste generation. In other embodiments, the separation chamber and one or more wires are constructed together. In addition, the one or more wires in the disclosed devices and methods allow for effective displacement-based separation and can meet or exceed the processing throughput or speeds of current commercial systems and devices.

In one aspect, a magnetophoretic separation device is disclosed that comprises a separation chamber comprising a plurality of channels that provide two or more streams. The streams comprise a sample stream comprising target biological materials and non-target biological materials, wherein the target biological materials are magnetically-labeled. The device further comprises a buffer stream that is substantially free of the sample in which the one or more streams combine in a single collection channel without fluidic mixing. The device also comprises one or more wire(s) carrying a current, the wires generating a magnetic force that deflects the one or more magnetically-labeled target biological materials into the buffer stream.

In one embodiment, the magnetophoretic separation of cells occurs through the use of an integrated electromagnet. In certain embodiments, the devices have a sheath-based design in which a system of two electromagnets acts cooperatively to displace cells within a central microfluidic channel. The devices disclosed herein can be used to isolate cells from both heterogeneous cell suspension in Newtonian fluids (e.g. saline) and non-Newtonian fluids (e.g., whole blood). The devices further allow parallel streams of liquid flowing in “laminar flow” (where the fluid streamlines move together without mixing, which occurs when the fluid flow rate is sufficiently slow). Magnetically-labeled cells are directed from the “sample” stream to the “collection” stream (i.e., buffer stream) by the action of the magnetic force. The selectivity is derived from the fact that only target cells are magnetically labeled using commercially-available antibody-coated magnetic particles.

In one embodiment, the applied magnetic field of this rational design is generated by an integrated electromagnet (current-carrying wire) located below the microfluidic channel. Electromagnets have several advantages over designs that utilize permanent magnets. For example, electromagnets can be easily switched on/off to facilitate cell capture and release. Second, the strength of the resultant magnetic field may be tuned by varying the current. In the microfluidic device context, electromagnets have seen limited use because they typically produce weak magnetic fields, and they generally require at least two steps of lithography that must be repeated in the fabrication of each device. In addition, the bulkiness of the electromagnet and potential Joule heating derived from large currents flowing through the electromagnet coil can become problematic. The device design described herein addresses these limitations by creating a new microfluidic device design derived from first-principles and rational design parameters.

The devices and methods described herein can be applied to diagnostic and regenerative medicine. Point-of-care diagnostic devices typically utilize a biological fluid sample analyte, such as lymph, blood, interstitial fluid, saliva, vaginal fluid, cellular material, or mucous fluid. Other sources of biological materials include but are not limited to peripheral whole blood, tissue digestate, amniotic fluid, umbilical cord blood, fine needle aspirates, vitreous humor biopsies, cerebrospinal fluid, or other biological fluids. In other embodiments, the target biological materials are cells, proteins, solutes, or particulates susceptible to a magnetic field.

To minimize contact with the analyte, or to minimize contaminating further tests, the microfluidic chamber can be disposable. Thus, the microfluidic component can be separated from the re-usable electromagnetic components of the design. In addition to addressing biohazard considerations, this arrangement will reduce costs associated with device manufacture and implementation.

The devices and methods disclosed can be used to separate cells of biomedical interest, which, despite their functional significance, are often present in very small numbers. For example, rare cells occur in a particular population in the range of rare cell type to other cell type in the ratio of about 1:100 to about 1:10⁹. Previously inaccessible rare cells include but are not limited to peripheral hematopoietic stem cells, endothelial progenitor cells, circulating tumor cells, mature circulating endothelial cells, amniotic stem cells, mesenchymal stem cells, adipose-derived stem cells, intestinal stem cells, skin stem cells, neural stem cells, cancer stem cells, adult stem cells, fetal stem cells, or progenitor cells. The analysis and isolation of rare cells require efficient and sensitive procedures that do not compromise the viability of the cells. In some embodiments, the disclosed devices and methods allow for the isolation of rare cells that preserve viability.

In another aspect, the disclosed methods for separating target biological materials from non-target biological materials in a sample comprise labeling the target biological materials in a sample with a magnetic tag and introducing the sample into at least a first inlet of at least a first channel in a magnetophoretic separation device. The methods also comprise introducing a buffer into a second inlet of a second channel of the magnetophoretic separation device and generating a magnetic force by providing a current in one or more wire(s) placed adjacent to the channels, thereby deflecting the labeled target biological materials into the channel carrying the buffer. The methods further comprise collecting the target biological materials from an outlet of the second channel.

In some aspects, the disclosed methods of constructing a magnetophoretic separation device comprise providing a substrate and constructing a separation chamber on the substrate, wherein the separation chamber comprises a plurality of channels, wherein one or more sample channels combine with a buffer channel in a single collection channel. The methods also comprise constructing one or more wire(s) carrying a current on the substrate adjacent to the separation chamber.

In some embodiments, the separation chamber can be constructed of a polymer (including but not limited to poly(dimethylsiloxane, cyclic olefin copolymer, polystyrene, and elastomer), a thermoplastic polymer (including but not limited to polypropylene, poly(methyl methacrylate (PMMA), and polycarbonate), glass, silicon, quartz, plastic (such as polyethylene), or other various suitable materials. The main characteristics of the material are that it is non-magnetic and microscale in size. The one or more wires used in the disclosed devices and methods can be a metal wire, such as a copper wire, mounted on a solid substrate, such as fiberglass reinforced epoxy laminates, including the print circuit board material called FR4. The construction of the one or more wires is not limited to copper on FR4 and can be printed on several different suitable platforms.

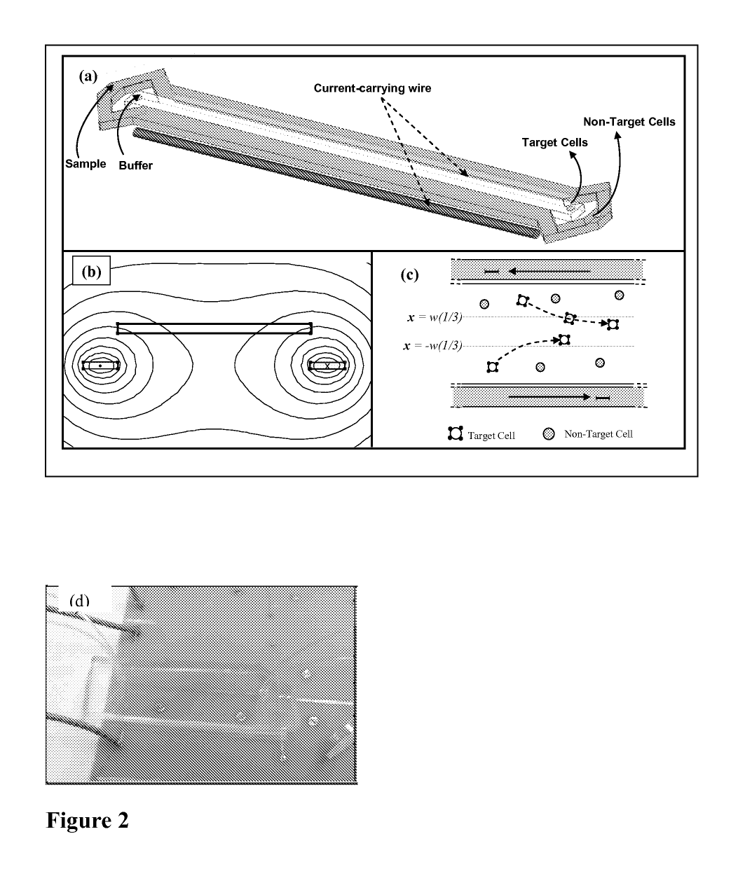

In certain embodiments, the device is first aligned with the current-carrying wire array, and the samples and buffer are injected into the corresponding inlets. In such embodiments, the device is designed to have a plurality of channels. The channels have outlets on opposite sides of the device. Target and non-target cells are collected from the correct outlets. The volumetric flow rate and applied current can be set as to ensure complete displacement of the target cells. FIG. 2 has been constructed assuming the target cells have been labeled with 1 um magnetic beads. Using Eq. [24], the characteristics of the beads can be evaluated to construct a new functional separation device. As shown in FIG. 2, the target cells displace into the center stream; therefore, the samples are injected at the set flow rate in the rear inlet and the buffer injected into the front inlet. Then the target cells can be collected in the front outlet and the non-target cells collected in the rear outlet (shown in FIG. 2A). The central rectangle represents fluid flow channel and shaded rectangles represent current-carrying wires. In some embodiments, the device includes an alignment guide that aligns the separation chamber with the one or more wires.

FIGS. 2A-D are schematic illustrations of one embodiment of the magnetophoretic separation design using dual wires. FIG. 2A is a graphical illustration of the dual-wire magnetophoretic separation device. FIG. 2B is a cross-sectional illustration of magnetic flux lines resulting from anti-parallel dual-wire configuration driving cell-particle complexes to the middle of device. FIG. 2C shows an injected sample split into two streams that sheath a central buffer stream. FIG. 2D is a photograph of a microfluidic chip with cell and buffer inlets (left), outlet tubing to collection tubes (right), and the chip aligned on the electromagnet wire array.

### Rational Design

The instant disclosure describes a rational design based on practical experimental constraints and the desired need for a microfluidic system capable of delivering both high efficiency and high purity. The described approach directly accounts for variations in key parameters within the cells, tagging particles, and device and addresses several key parameters, which allows this device to be used in clinical and bench-top applications. Although many modeling approaches have been well established in the literature, these approaches fail to address all of the requirements of a clinically-usable cell separation platform. The magnet-based biological material separation device disclosed incorporates clinical diagnostic considerations ab initio by constraining the device microfluidic channel dimensions to a practical scale (i.e., that of a microscope slide) and incorporating disposable and non-disposable components (fluidic part and magnetic part, respectively) in the device. Furthermore, the incorporation of a tunable electromagnet (relative to state-of-the-art on-chip designs that employ permanent magnets) maximizes versatility in addition to reducing device cost. In addition, the design accounts for drag forces experienced by cells tagged with hundreds of magnetic beads. This approach is more realistic for continuous-flow cell separation compared to that described by prior theoretical/computational models that only consider the manipulation of magnetic micro- or nanoparticles in the absence of cell attachment, as cells are generally much larger in size relative to the particles.

Furthermore, embodiments of the disclosed magnetophoretic microfluidic device designs and methods use optimized dimensions and operating conditions. The microfluidic devices disclosed herein were designed using computational based design on a force balance equation that considers the two driving forces exerted on a magnetically-tagged cell moving through a Newtonian liquid. The main forces considered are the magnetic forces \vec{F}ₘ originating from a current-carrying wire located adjacent to the device to draw a tagged particle towards a desired location and the Stokes force \vec{F}ₛ that opposes the motion of the particle. The variables associated with the described derivations are provided in the list below.

h = Height
k = thermal conductivity
l = Length
q = Joule Heat
t = Time
w = Width
x = Distance
A = Cross-sectional area
B = Magnetic field
F = Force
I = Current
H = Coverslip height
L = Coverslip length
R = Radius
T = Temperature
V = Volumetric flow rate
W = Coverslip width
χ = Susceptibility
η = Viscosity
μₒ = Permeability in vacuum
ρ_{M} = Density
ρ_{R} = Electrical resistivity
φ = Number of particle attached to cell
Subscripts
c = Cell
ch = Channel
inj = Injection
m = Magnetic
p = Particle
s = viscous drag (Stokes Law)
w = Wire
x = vector in the x-direction
y = vector in the y-direction
z = vector in the z-direction

An experimental analysis of several of the key parameters associated with magnetophoretic devices has been performed (i.e. magnetic particle and cell properties, and cell-particle binding characteristics) and directly accounted for in the resulting computation. Several different width designs were fabricated and experimentally validated against the derived optimization, whereas applied current and flow rates were tuned and compared with the rational design, as to provide a functional cell separation platform.

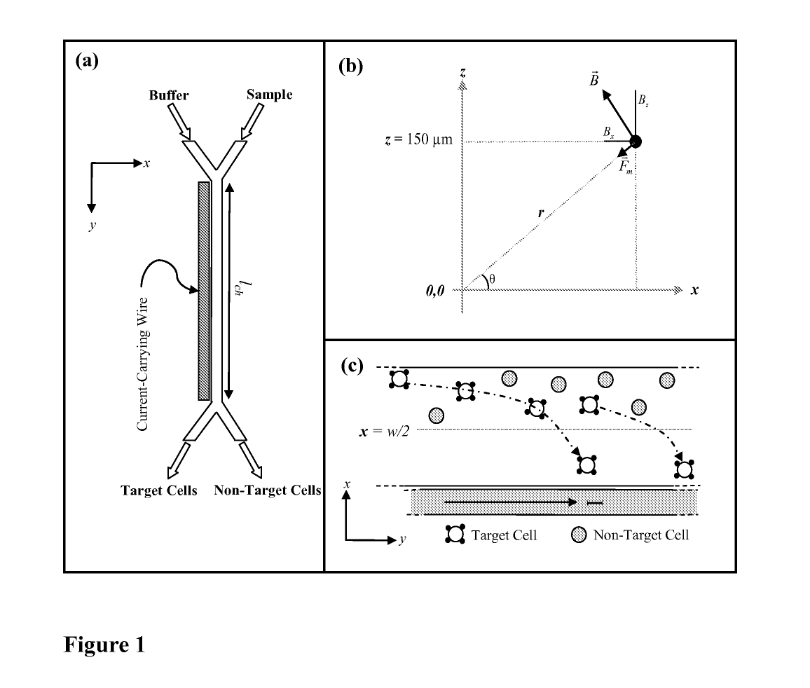

In some embodiments, the devices comprises a single wire. In other embodiments, the device comprises two or more wires. FIGS. 1A-C are schematic illustrations of one embodiment of the disclosed magnetophoretic separation device using a single wire. FIG. 1A illustrates the configuration of a single current-carrying wire as part of the magnetophoretic separation device. In FIG. 1A, a buffer stream is injected on the side closest to the current-carrying wire, and a sample stream is injected to the far-side with respect to the current-carrying wire. The device length required for the target cell to displace from the sample stream to the buffer stream is l_{ch}. FIG. 1B shows a mathematical configuration of a single current-carrying wire located at (0,0) with current flowing in the positive y-direction (out of the page). The particle is positioned at a height above of the wire (z=150 μm) and a distance away (x) from the current-carrying wire. The magnetic force vector (\vec{F}ₘ) perpendicular to direction of the magnetic field vector (\vec{B}). (FIGS. 1A and 1C). FIG. 1C is a schematic illustration of the separation device displacing target cells from the sample stream into the buffer stream, while non-target cells remain in the sample stream. The distance required for complete displacement of target cells from the far-edge to interface of the sample and buffer streams is half the width of device (x=w/2).

In other embodiments, the device comprises a dual-wire configuration. The incorporation of a second conducting wire in parallel alignment with the first wire allows tagged-cell displacement in both positive and negative lateral (\hat{x}) direction towards a center stream of buffer. This design reduces the displacement distance required for cell isolation by increasing the magnetic forces experienced by the cell-particle complex. As shown in FIG. 2A, the current-carrying wires of the device are located equidistant from the center of the flow channel and the two currents are assumed to run anti-parallel in the ŷ-direction, parallel to the fluidic flow of the chamber. The microfluidic device is separated from the current-carrying wire array by a vertical distance ranging from about 10 microns to about 500 microns. In other embodiments, the vertical distance ranges from about 20 microns to about 480 microns, from about 40 microns to about 440 microns, from about 60 microns to about 460 microns, from about 80 microns to about 440 microns, from about 100 microns to about 420 microns, from about 120 microns to about 400 microns, from about 140 microns to about 380 microns, from about 160 microns to about 360 microns, from about 180 microns to about 340 microns, form about 200 microns to about 320 microns, from about 220 microns to about 300 microns, from about 240 microns to about 280 microns. In other embodiments, the vertical distance is about 100 microns, about 125 microns, about 150 microns, about 175 microns, or about 200 microns.

### Theoretical Considerations of Microfluidic Designs

While this disclosure is not bound by any theories, this section describes the derivation of an expression for the displacement of a magnetic particle in a channel subjected to both magnetic \vec{F}ₘ and Stokes \vec{F}ₛ forces. The magnetic force and the Stokes force contributions are considered separately; note that gravity and buoyancy forces are negligible and are thus not considered here. The effect of Joule heating is also considered in this section. Prior to derivation of the expected cell displacement under these two driving forces, the potential role of diffusion was examined. Using the Stokes-Einstein relation,

$${D}_{AB} = \frac{{k}_{B} T}{6 π η {R}_{c}} ,$$

and the random walk theory for displacement in one dimension, Δx≈(2D_{AB}t)^{0.5}, the diffusivity (D_{AB}) of an average cell in buffer can be derived, as well as the transverse displacement (Δx) within the microfluidic device. It can be shown that a cell would have a diffusive constant (D_{AB}) on the order of 10⁻¹⁵ m² s⁻¹ at room temperature. Assuming a channel with dimensions of 5 (L)×0.2 (W)×0.05 (H) cm and a suspension flow rate of 10 μL min⁻¹, the residence time (t) of a cell within the channel is 2.5 min, with lateral (Δx) diffusion of less than 300 nm. Therefore, it was concluded that the effect of diffusion may be ignored within the described design.

### A. Magnetic Force Determination

The trajectory of a magnetically-labeled cell in the proposed microfluidic device is modeled by evaluating the forces on the cell generated by motion through the fluid under the attractive action of a magnetic field. Prior to derivation of forces on a cell-particle complex, the forces specific to a single magnetic particle are determined. In the following discussion, the particle is initially located at position (x,y,z), subjected to a magnetic field \vec{B} originating from a current-carrying wire at (0,0,0), as shown in FIG. 1B. The particle is fixed at z=150 μm and moves laterally in the −x-direction, towards to the current-carrying wire independent of the y-component.

A single magnetic particle is idealized as a magnetic sphere of uniform moment density. The magnetic force exerted on the particle, \vec{F}ₘ=(\vec{m}·∇)\vec{B}, may be evaluated from the total moment on the particle \vec{m}=Vₚ\vec{M} which depends on the volume of the particle (Vₚ) and the volume magnetization \vec{M} Here, \vec{M}=Δχ\vec{H} and Δχ is the volumetric magnetic susceptibility difference between the particle (χₚ) and the surrounding buffered fluid medium (χ_{med}). The overall response of a magnetic particle in a fluid to a magnetic field is then determined by the strength and gradient of the applied magnetic field (\vec{B}=μₒ\vec{H}), yielding:

$${\vec{F}}_{m} = \frac{{V}_{p} {Δ}_{χ}}{{μ}_{o}} ( \vec{B} \cdot ∇ ) \vec{B} \qquad Eq . [ 1 ]$$

where μₒ is the permeability of vacuum equal to 4π×10⁻⁷ T m A⁻¹. It should be noted that a complementary form of this equation can be determined be applying the Maxwell equation ∇×\vec{B}=0 to the following mathematical identity:

∇(\vec{B}·\vec{B})=2\vec{B}×(∇×\vec{B})+2(\vec{B}·∇)=2(\vec{B}·∇)\vec{B} Eq. [2]

Therefore, Eq. [1] can alternatively be expressed as:

$${\vec{F}}_{m} = {V}_{p} {Δ}_{χ} ∇ ( \frac{{\vec{B}}^{2}}{2 {μ}_{o}} ) \qquad Eq . [ 3 ]$$

Although Eq. [3] is a valuable relationship for visualizing the magnetic force operative in the system, all subsequent \vec{F}ₘ analyses are derived from relationships expressed in Eq. [1]. As the magnetic susceptibility of the surroundings is typically 5-6 orders of magnitude lower than that of the particles, Δχ is determined primarily by the susceptibility of the particle, χₚ. By way of example, the magnetic susceptibility of phosphate buffer saline is on the order of 10⁻⁷ and that of blood is on the order of 10⁻⁶, while the susceptibility of commercial magnetic oxide particles is generally on the order of 10⁰-10⁻¹. Furthermore, it should be noted that the magnetic susceptibility of materials commonly used in the construction of a microfluidic channel has also been found to be several orders of magnitude smaller (approximately 10⁻⁵-10⁻⁶) than that of the magnetic beads, and thus the effect of the device itself may also be assumed to be negligible in this analysis.

With these considerations, Eq. [1] can then be expanded in explicit form to yield:

$$\begin{matrix}{\vec{F}}_{m} = \frac{{V}_{p} {χ}_{p}}{{μ}_{o}} ( \vec{B} \cdot ∇ ) \vec{B} \\ = \frac{{V}_{p} {χ}_{p}}{{μ}_{o}} [ \begin{matrix}{B}_{x} \frac{\partial {B}_{x}}{\partial x} + & {B}_{y} \frac{\partial {B}_{x}}{\partial y} + & {B}_{z} \frac{\partial {B}_{x}}{\partial z} \\ {B}_{x} \frac{\partial {B}_{y}}{\partial x} + & {B}_{y} \frac{\partial {B}_{y}}{\partial y} + & {B}_{z} \frac{\partial {B}_{y}}{\partial z} \\ {B}_{x} \frac{\partial {B}_{z}}{\partial x} + & {B}_{y} \frac{\partial {B}_{z}}{\partial y} + & {B}_{z} \frac{\partial {B}_{z}}{\partial z}\end{matrix} ]\end{matrix} \qquad Eq . [ 4 ]$$

The above equation can be simplified by assuming the current-carrying wire located at the side of the device is infinitely long in the ŷ-direction, thus allowing the spatial derivatives of the magnetic field to vanish:

$$\frac{\partial \vec{B}}{\partial y} = 0 ⇒ \{ \frac{\partial {B}_{x}}{\partial y} = 0 , \frac{\partial {B}_{y}}{\partial y} = 0 , \frac{\partial {B}_{z}}{\partial y} = 0 \} \qquad Eq . [ 5 ]$$

Furthermore, the symmetry of the device design (Section II) dictates that there are no off-axis components of the magnetic field gradient:

$$\{ \frac{\partial {B}_{y}}{\partial x} = 0 , \frac{\partial {B}_{y}}{\partial z} = 0 \} \qquad Eq . [ 6 ]$$

In one embodiment, a single, rectangular current-carrying wire is placed at the periphery of the device (FIG. 1). The wire is situated below the microchannel and is kept separate from the fluidic separation channel. In FIG. 1A, a buffer stream is injected on side closest to current-carrying wire and sample stream injected to the far-side with respect to the current-carrying wire. The device length required for the target cell to displace from the sample stream to the buffer stream is l_{ch}. FIG. 1B shows a mathematical configuration of single current-carrying wire located at (0,0) with current flowing in the positive y-direction (out of the page). The particle positioned at height above of the wire (z=150 μm) and distance away (x) from the current-carrying wire. The magnetic force vector (\vec{F}ₘ) perpendicular to direction of the magnetic field vector (\vec{B}). In FIGS. 1A and 1C, the cell separation device displaces target cells from sample stream to buffer stream; non-target cells remain in sample stream. The distance required for complete displacement of target cells from the far-edge to interface of the sample and buffer streams is half the width of device (x=w/2).

Employing the Biot-Savart Law the magnetic field E at a distance (r) from the current-carrying wire can be determined in cylindrical coordinates as:

$$\vec{B} = \frac{{μ}_{o} I}{2 π r} \hat{θ} \qquad Eq . [ 7 ]$$

For a single current-carrying wire located at the origin (0,0,0) in FIG. 1 with current flowing in the positive ŷ-directions (out of the page), Eq. [7] is explicitly written in Cartesian coordinates as:

$${B}_{x} = - \frac{{μ}_{o} I}{2 π r} \frac{z}{r} = \frac{{μ}_{o} I}{2 π} \frac{z}{{x}^{2} + {z}^{2}} \\ {B}_{z} = \frac{{μ}_{o} I}{2 π r} \frac{x}{r} = \frac{{μ}_{o} I}{2 π} \frac{{x}^{2}}{{x}^{2} + {z}^{2}} \qquad Eq . [ 8 a \text{-} b ]$$

The magnetic force in the tangential direction is provided as:

$${F}_{m , x} = \frac{{V}_{p} {χ}_{p}}{{μ}_{o}} [ {B}_{x} \frac{\partial {B}_{x}}{\partial x} + {B}_{z} \frac{\partial {B}_{x}}{\partial z} ] \qquad Eq . [ 9 ]$$

The x- and z-components of the gradient of the magnetic field are listed below:

$$\frac{\partial {B}_{x}}{\partial x} = \frac{{μ}_{o} I}{2 π} \frac{2 zx}{{[ {x}^{2} + {z}^{2} ]}^{2}} \\ \frac{\partial {B}_{x}}{\partial z} = - \frac{{μ}_{o} I}{2 π} \frac{{x}^{2} - {z}^{2}}{{[ {x}^{2} + {z}^{2} ]}^{2}} \qquad Eq . [ 10 a \text{-} b ]$$

and are substituted back into Eq. [9] to obtain the expression for the magnitude of the magnetic force exerted on a magnetic particle under the above stated conditions:

$${F}_{m , x} = - \frac{{V}_{p} {χ}_{p}}{{μ}_{o}} [ {( \frac{{μ}_{o} I}{2 π} )}^{2} \frac{{x}^{2}}{{( {x}^{2} + {z}^{2} )}^{2}} ] \qquad Eq . [ 11 ]$$

### B. Hydrodynamic Resistance Force Determination

In addition to the magnetic force \vec{F}ₘ acting on the magnetic particles, there exists a viscous drag force \vec{F}ₛ acting on the particle in the direction opposite to the particle motion. This drag force, or Stokes force \vec{F}ₛ, is a function of the suspension medium viscosity, the radius of the particle (Rₚ), and the velocity (\vec{v}) of the particle in the direction of the magnetic force. The inertial effects on the particles suspended in the fluid are negligible, as the Reynold's number is less than unity due to the geometric constraints of the microfluidic chamber. This result implies that while the fluid exerts drag on each particle, the particles exert no force on the fluid. Furthermore, the number of particles within the suspension is assumed dilute, thus there exist no short-range inter-particle dipolar interaction. As the magnetic particle is carried by the flowing solution in the ŷ-direction, within the context of laminar flow, the hydrodynamic forces only act in the \hat{x}-direction (direction perpendicular to flow). This force can be expressed as:

\vec{F}ₛ=6πηRₚv\hat{x} Eq. [12]

### C. Joule Heating

Resistive heating of the wires with respect to time (\dot{q}), or Joule heating, may limit the applied current range and geometry of the wires. Furthermore, this effect can adversely affect the flow and character of the fluid and may even degrade the device, rendering it non-reusable. Joule heating is determined by both the current conductor geometry and by the time duration of the applied current; the heat generated in this manner is given by:

\dot{q}=I²R Eq. [13]

Assuming no dissipative cooling, the electric resistance is equal to

$$R = \frac{{ρ}_{R} {l}_{w}}{A}$$

for small wires, where l is the length of the wire, A the cross-sectional area and ρ_{R} the electrical resistivity. This heat loss is dependent upon wire surface area, the thermal properties of materials of construction, and the properties of the ambient surroundings. Therefore, while current flow is constantly heating the wire as described in Eq. [13], the wire is simultaneously cooling through heat transfer to the surroundings (via radiation, convection, and conduction through its surface and to the surroundings). Although all three heat transfer phenomena are occurring simultaneously, most of the energy transfer is via conduction through the substrate containing the wires and through the microfluidic device above the wires. Hence, the general Fourier's Law of heat conduction equation (or heat loss) appropriate for this situation is that mimicking an anisotropic medium, which is given as:

$${k}_{x} \frac{{\partial }^{2} T}{\partial {x}^{2}} + {k}_{y} \frac{{\partial }^{2} T}{\partial {y}^{2}} + {k}_{z} \frac{{\partial }^{2} T}{\partial {z}^{2}} + \dot{q} = {ρ}_{M} {C}_{P} \frac{\partial T}{\partial t} \qquad Eq . [ 14 ]$$

where kₓ, k_{y}, and k_{z} are the thermal conductivities of the substrate in the \hat{x}-, ŷ- and \hat{z}-direction, respectively, \dot{q} is the heat generation term (or Joule heating), and ρ_{M} and Cₚ are the density and heat capacity of the substrate, respectively.

Under the assumption that the width and length of the substrate are much larger than the height, the heat transfer in the \hat{x}- and ŷ-direction can be ignored, thus

$$\frac{d T}{d x} = \frac{d T}{d y} = 0.$$

Combining Eq. [14] and Eq. [13] yields:

$$- {k}_{z} \frac{\partial T}{\partial z} = {\dot{q}}_{o} g ( t ) f ( x ) \qquad Eq . [ 15 ]$$

where g(t) defines the time varying characteristics of the current that generates the heat, f(x) is a spatial function that defines the wire locations along the \hat{x}-direction, and qₒ is the Joule heating provided at a reference current. Both g(t) and f(x) are controlled by the current characteristics and wire geometry, respectively. It should be noted that for the case of direct current (DC), g(t)=1 and \dot{q}ₒ=\dot{q}. To complete the problem statement, it is assumed that the initial temperature is set equal to room temperature (298 K). To obtain the steady-state solution for a set temperature rise as a function of current, 4 and wire cross-sectional area, A, a classical Fourier series method is needed. Such calculations are well established and known to those of skill in the art.

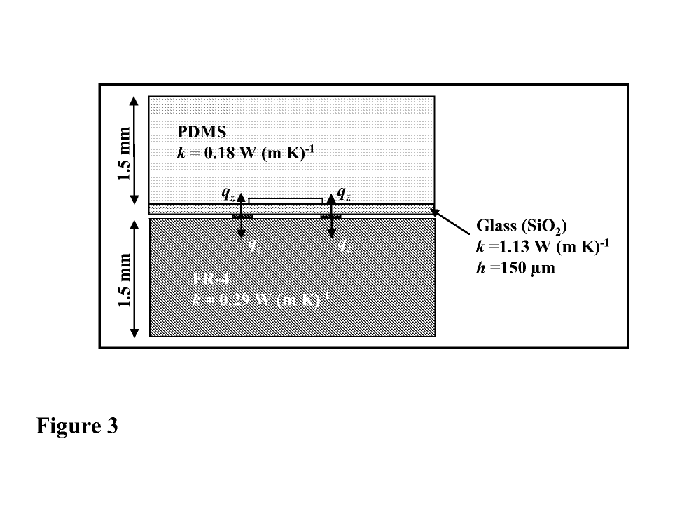

For the Joule heating calculation, the following physical characteristics are assumed for the device. Briefly, the overall layout of the biological material separation device includes, for example, a thick poly(dimethylsiloxane) (PDMS) slab bound to a thin glass coverslip, which is mounted on thin current-carrying copper wires deposited on a printed circuit board (PCB) substrate (FIG. 3). FIG. 3 is a schematic illustration of a cross-section of a printed circuit board electromagnetic array along with PDMS microfluidic device used in evaluation of Joule heating constraints within rational device design. First, both FR-4 and PDMS are assumed to have a thermal conductivity of ˜0.2 W (m K)⁻¹ and heat transfer is assumed in z-direction only. The glass coverslip employed in investigation is only 150 μm thick (h), and, assuming minimal contact with air, thermal resistance above copper wires (brown) assumed equal to PDMS alone.

In some embodiments, PCB copper wire arrays are mounted onto substrates composed of a material known as FR-4, which is a woven fiberglass cloth bound with an epoxy resin. For the purposes of this device model, the thermal conductivities of the PDMS and FR-4 were assumed to be equal to 0.2 W (m K)⁻¹ and of equal thicknesses (1.5 mm), with the thermal resistance of the glass coverslip ignored. Solving Eq. [15] at the steady state condition, the current carrying capacity, or the maximum current Iₘₐₓ which results in a specified temperature increase, was computed to be approximately Iₘₐₓ=2A for a nominal temperature rise of 20 K and for a standard PCB copper wire array cross-section of 35 μm×178 μm. Therefore, in the present experimental set-up, Joule heating only becomes a concern at high currents (I>2 A) and/or if the wire cross-sectional area is significantly reduced.

### IV. Optimization of the Channel Length Device Designs

Utilizing the viscous drag \vec{F}ₛ, magnetic force \vec{F}ₘ, and Joule heating results obtained from earlier sections, it is now possible to explore the performance of the device subjected to realistic parameter values. It is desired to create a microfluidic cell separation device that delivers the greatest lateral displacement in the shortest possible channel, i.e. maximize x and minimize channel length l_{ch}. The overall force on the magnetic particle is the sum of the magnetic force \vec{F}ₘ (Section III.A.) and the hydrodynamic force \vec{F}ₛ (Section III.B.) that lends a constant velocity to the particle, which explicitly sets the acceleration equal to zero:

\vec{F}ₘ+\vec{F}ₛ=0 Eq. [16]

Eq. [16] yields the overall equation representing the force in the x-direction exerted on a magnetic particle:

$$- \frac{{V}_{p} {𝒳}_{p}}{{μ}_{o}} [ {( \frac{{μ}_{o} I}{2 π} )}^{2} \frac{{x}^{2}}{{( {x}^{2} + {z}^{2} )}^{2}} ] - 6 πη {R}_{p} {v}_{x} = 0 \qquad Eq . [ 17 ]$$

The force balance can now be rearranged and solved for an optimized channel geometry to obtain a magnetophoretic microfluidic device design, under the assumption of fully-developed fluid flow.

### Microfluidic Device Design

Rearranging Eq. [17] and assuming spherical geometry for the magnetic particles

$$( {V}_{p} = \frac{4}{3} π {R}_{p}^{3} )$$

allows determination of the velocity \vec{v}, normal to the magnetic field \vec{B}, in the \hat{z}-direction, of a magnetic particle flowing in the device channel. This process also allows an estimate of the time t for the particle to traverse a given distance across the device channel width. In accordance with the geometry of the system, the velocity of the particle in the \hat{x}-direction is provided as:

$${v}_{x} = \frac{d x}{d t} = - \frac{{R}_{p}^{2} {𝒳}_{p} {μ}_{o} {T}^{2}}{18 {ηπ}^{2}} [ \frac{x}{{( {x}^{2} + {z}^{2} )}^{2}} ] \qquad Eq . [ 18 ]$$

The variables in Eq. [18] are: Rₚ the radius of the magnetic particle, χ_{b} the volumetric susceptibility of the particle, I the current applied to the wire, η the viscosity of the carrier solution, and z the gap distance fixed by the glass coverslip. It should be noted that realization of an actual microfluidic magnetophoresis device requires the successful attachment of a large number of magnetic particles to the target cells in order to convey the largest magnetic moment possible to the travelling complex. In the case of such a cell-particle complex, the above analysis is altered slightly to account for the drag forces on the cell itself, a body that is one to two orders of magnitude larger than the microparticles alone, depending on the actual tagging cell-particle characteristics. Therefore, the viscous drag force on the cell-particle complex is now calculated using the larger cell radius (R_{c}), allowing the magnetic particle radius to be neglected (R_{c}>>Rₚ). The actual magnetic force exerted on a labeled cell derived from the magnetic field of the current-carrying wire is equal to the magnetic force on one particle (as described in Eq. [11]) multiplied by the number of particles attached to the cell φ, or \vec{F}ₘ·φ. Substitution of these parameters into Eq. [18] allows an estimate for the displacement time of the cell-particle complex out of the stream into the buffer fluid, attracted to the current-carrying wire:

$${v}_{x} = \frac{d x}{d t} = - \frac{{R}_{p}^{3} {φ𝒳}_{p} {μ}_{o} {I}^{2}}{18 {R}_{c} {ηπ}^{2}} [ \frac{x}{{( {x}^{2} + {z}^{2} )}^{2}} ] \qquad Eq . [ 19 ]$$

The above differential equation may be solved analytically to obtain a solution of the form x=f(t), where f(t) is the residence time of a cell of radius R_{c} the microfluidic chamber. In this manner, the lateral displacement x in the device of the magnetic cell-particle complex for any given value of time t can be calculated. This quantitative estimation of the physical parameters of the cell-particle complex flowing in a laminar fashion through the designated microfluidic device allows determination of the optimal dimensions of the device. In particular, specification of the cell-particle complex displacement time t required to traverse the channel width w permits determination of the minimum length (l_{ch}) of channel. Input of specific known parameters such as the input volumetric flow rate (\dot{V}) of the carrier fluid, the device channel height (h) and width (w) of the carrier fluid stream leads to calculation of the optimum length of the proposed device. The calculated residence time t may then be translated to a calculation of the lengthwise displacement l_{ch} of the cell-particle complex as the volumetric flow rate, \dot{V}, is given by:

$$\dot{V} = \frac{V}{t} = \frac{{l}_{ch} \cdot w \cdot h}{t} \qquad Eq . [ 20 ]$$

where l_{ch} represents the distance traveled along the channel along the ŷ-direction (lengthwise). Again, the microfluidic device design objective is to deliver the greatest lateral displacement in the shortest possible channel, or in other words, to maximize x whilst minimizing l_{ch}. To this end, the solution of Eq. [20] for residence time t and subsequent incorporation of Eq. [19] yields a relationship for the lateral particle displacement as a function of the distance travelled along the channel, x=f(l_{ch}), under the influence of an applied magnetic field, with the trajectory provided below:

$$\frac{d x}{d {l}_{ch}} = - \frac{{R}_{p}^{3} φ wh 𝒳 {μ}_{o} {I}^{2}}{18 {R}_{c} \dot{V} {ηπ}^{2}} [ \frac{x}{{( {x}^{2} + {z}^{2} )}^{2}} ] \qquad Eq . [ 21 ]$$

In some embodiments, the microfluidic device has two conducting wires in parallel configuration to each other. To determine the resultant magnetic field generated by the two conducting strips in the Generation II design, improving upon the single-wire design of Generation I, an array of conductors is considered where one conductor is positioned at far edge of the microfluidic channel with current flowing in the negative ŷ-direction and the other conducting wire at the alternate edge of the microfluidic channel with current in the positive ŷ-direction, as shown in FIG. 2. The magnetic field components at any point (x,z) resulting from current flowing through the two conductors, set a distance equal to 2× apart, are given as:

Bₓ(x,z)=Bₓ^{o}(x−X,z)−Bₓ^{o}(x+X,z)

B_{z}(x,z)=B_{z}^{o}(x−X,z)−B_{z}^{o}(x+X,z) Eq. [22a-b]

where Bₓ^{o} and B_{z}^{o} are the field components determined for the single-wire (Generation I) configuration as specified in Eq. [8a-b] (as described in Section III. A.). Insertion of Eq. [22a-b] into the magnetic force equation (Eq. [9]) yields the expression for the magnitude of the attractive force exerted on a magnetic particle in the double-wire sheath (Generation II) device design configuration:

$${F}_{m , x} = \frac{{V}_{p} {𝒳}_{p}}{{μ}_{o}} [ ( {B}_{x} ( x - X , z ) - {B}_{x} ( x + X , z ) ) ( \frac{\partial }{\partial x} \{ {B}_{x} ( x - X , z ) - {B}_{x} ( x + X , z ) \} ) + ( {B}_{z} ( x - X , z ) - {B}_{z} ( x + X , z ) ) ( \frac{\partial }{\partial z} \{ {B}_{x} ( x - X , z ) - {B}_{x} ( x + X , z ) \} ) ] \qquad Eq . [ 23 ]$$

Substituting this new equation into Eq. [16], which describes the overall force balance, and rearranging as outlined above in Eq. [18-21], yields an equation for displacement as a function of channel length in the double-wire (sheath) device design:

$$\frac{d x}{d {l}_{ch}} = \frac{{R}_{p}^{3} φ wh {𝒳}_{p} {μ}_{o} {I}^{2}}{18 {R}_{c} \dot{V} {ηπ}^{2}} [ ( \frac{z}{{( x - X )}^{2} + {z}^{2}} - \frac{z}{{( x + X )}^{2} + {z}^{2}} ) ( - \frac{2 z ( x - X )}{{[ {( x - X )}^{2} + {z}^{2} ]}^{2}} + \frac{2 z ( x + X )}{{[ {( x + X )}^{2} + {z}^{2} ]}^{2}} ) + ( - \frac{( x - X )}{{( x - X )}^{2} + {z}^{2}} + \frac{( x + X )}{{( x + X )}^{2} + {z}^{2}} ) ( \frac{{( x - X )}^{2} - {z}^{2}}{{[ {( x - X )}^{2} + {z}^{2} ]}^{2}} - \frac{{( x + X )}^{2} - {z}^{2}}{{[ {( x + X )}^{2} + {z}^{2} ]}^{2}} ) ] \qquad Eq . [ 24 ]$$

The resultant differential equation cannot be solved analytically. However, a solution of the form l_{ch}=f(x) can be obtained by numerical integration using the 5^{th}-order Runge-Kutta method and appropriate solver software, such as MATLAB®.

It can be shown that by adding one additional wire to the array increases the magnetic force by at least a factor of four. Using Eq. [3] and the geometry illustrated in FIG. 2, it can be seen that the magnitude of the magnetic field \vec{B} would be doubled, increasing the magnetic force \vec{F}ₘ by a factor of four.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

### EXAMPLES

### Example 1

### Microfluidic Device Design

The following examples are provided to illustrate, not limit, the invention. Example 1 describes the design of magnetophoretic separate devices for the separation of target biological materials, as well as methods of separation using the disclosed devices.

The derivation of the motion of a cell-particle complex in the described microfluidic design was completed by determining realistic values for applicable parameters within the mathematical expressions. To this end, the attainment of reasonable values for cell radius (R_{c}), magnetic susceptibility (χ), and particle binding characteristics (φ) was achieved via experiments conducted on a breast cancer cell line that served as a model for metastatic tumor cells.

### Materials and Instrumentation

Dynabeads® MyOne™ superparamagnetic microbeads (Invitrogen, Carlsbad, Calif.) were used as the model tagging particles to bind to the cells. These beads are composed of highly cross-linked polystyrene with superparamagnetic magnetite nanoparticles embedded within their matrices. These microbeads are coated with carboxylic acid (—COOH) groups that allow for the conjugation of biomolecules to their outer surface. According to the manufacturer, the magnetic particle diameter is 1.05±0.03 μm (Rₚ=0.525±0.015 μm), the magnetic mass susceptibility is 85×10⁻⁵ m³ kg⁻¹, and the density is 1800 kg/m³, which corresponds to a volume magnetic susceptibility χ=1.5. An approximate particle concentration value of 7−12×10⁹ particles per mL of stock particle suspension was also provided by the manufacturer. However, it should be noted that all these parameters may vary across lots and must be validated experimentally, as described below, to ensure a realistic rational device design. Quantitative results of the key parameters necessary to determine the device performance are provided in the last part of this section.

### Model Cell Culture Conditions and Characteristics

MCF-7 human breast adenocarcinoma cells (ATCC, Manassas, Va.) were cultured in 75 cm² tissue culture flasks at 37 C in a humidified atmosphere with 5% CO₂ and 95% air. The cells were incubated in Eagle's Minimum Essential Medium (EMEM; ATCC) supplemented with 10% fetal bovine serum, 100 U mL⁻¹ penicillin, 100 μg mL⁻¹ streptomycin and 0.01 mg mL⁻¹ bovine insulin. Cells were grown to pre-confluence and isolated for experiments by trypsinization using a 0.25% Trypsin-EDTA solution.

The average cell radius was determined via electronic volume using a Coulter counter (Cell Lab Quanta™ SC; Beckman Coulter, Brea, Calif.) and compared to size-calibration beads (Flow-Check™ Fluorospheres; Beckman Coulter). The resulting average radius was also validated by bright-field microscopy with manual assessment of the radius of a number of the cells.

### Magnetic Particle Diameter and Characteristics

The magnetic microbead radius was determined via field emission-scanning electron microscopy (FE-SEM; Hitachi S4800, Peoria, Ill.) of a dried particle suspension. Dried stock suspension was mounted on aluminum stubs and sputter-coated with gold-palladium to 2 nm thickness to provide a connection path for electron density in FE-SEM examination. The experimentally-determined particle concentration of the stock solution was verified against the concentration provided by the manufacturer. The stock suspension of particles was diluted 10,000× and counted using a hemacytometer and a Nikon TE2000 Inverted Microscope employing Nikon Elements Advance Research software.

The magnetic susceptibility of the polymer/magnetite beads was confirmed via superconducting quantum interference device (SQUID; Quantum Design MPMS XL-5, San Diego, Calif.) magnetometry. A 2 μL droplet of stock suspension was dried on a formvar-coated copper transmission electron microscopy grid (Electron Microscopy Science, Hatfield, Pa.). Magnetic hysteresis loops were measured at 300 K in the field range −5 kOe>H>5 kOe. The moment of a blank grid was also measured and subtracted from the measured data; data were normalized to the mass of particles. The magnetic character obtained from three replicates was averaged and the volumetric susceptibility was determined using the density values for the particles provided by Invitrogen.

### Particle-Cell Attachment Density

A binding assay was conducted to determine the number of particles that can attach to MCF-7 cells. DynaBeads® MyOne™ Carboxylic Acid particles were modified with antibodies against the epithelial cell adhesion molecule (anti-EpCAM; Santa Cruz Biotechnology, Santa Cruz, Calif.) using standard carbodiimide chemistry in ratios suggested by the carbodiimide coupling reagent manufacturer (1:1 molar ratio of beads to protein; Pierce Biotechnology, Rockford, Ill.). Modified particles were incubated with approximately 1×10⁶ cells for 30 min. in 1 mL EMEM at concentrations of 0.1 mg mL⁻¹, 0.5 mg mL⁻¹, and 1 mg mL⁻¹. Following incubation, the cell-particle complexes were removed from suspension using a permanent magnet and were then incubated with a fluorescently-labeled antibody against EpCAM (anti-EpCAM-FITC; Santa Cruz Biotechnology) for 30 min. at a concentration of 1:100 (v/v). A cell suspension containing zero particles was also incubated with anti-EpCAM-FITC at the same concentration for comparison. In both cases cells were separated from suspension via centrifugation at 190×g for 5 min. The supernatant was retained and dried in order to assess the mass of particles that remained after tagging, representing the unbound particles. All fluorescently-tagged cells were re-suspended in phosphate buffered saline (PBS) and subsequently analyzed for available receptor densities using flow cytometry. Results concerning the number of free receptors were compared with an antibody binding capacity calibration curve (Quantum™ MESF Beads; Bang Laboratory, Fishers, Ind.) to determine the initial number of available receptors on each cell (no particle attachment) and the number of receptors remaining after particle incubation (post-particle attachment). Comparison of the unbound particle mass, along with data obtained from untagged particle densities and the remaining receptor number after particle incubation, allows for estimation of the average particle density on an individual cell.

### Fabrication of a Validation Design

To validate the optimized device design, microfluidic channels were fabricated as previously described. To form the polymeric chambers, poly(dimethylsiloxane) (PDMS; Sylgard 184, Dow Corning, Midland, Mich.) elastomer was mixed (10:1 ratio) and poured onto a negative master, degassed, and allowed to cure overnight. PDMS replicas were then removed; inlet and outlet holes were punched with a 19G blunt-nose needle. Replicas and glass coverslips (60 (L)×24 (W)×0.15 (H) mm³) were then exposed to oxygen plasma and placed in contact to bond irreversibly.

Wire arrays were designed using PCB123® printed-circuit board design software and ordered from Sunstone Circuits (Mulino, Oreg.). The wire dimensions were set to provide a gap encompassing the width of the device microfluidic channel; the height and width of the all of the wires were set to 35 μm and 178 μm, respectively. Teflon-insulated 18G copper wires were soldered to the ends of each of the printed circuit board arrays and the arrays were connected to a DC power supply (Elenco Electronics XP-4, Wheeling Ill.) that provided three fixed-current setting at 0.25 A, 0.50 A and 1.00 A via standard alligator clip connectors. The PDMS channels and wire arrays were visually aligned followed by injection of a prepared homogenous MCF-7 cell suspension using a syringe pump (Harvard Apparatus, Holliston, Mass.).

### Results

To ensure a robust microchannel device, several measurements of each parameter were performed to obtain meaningful values for key parameters associated with the proposed device designs. These average values are presented in Table 1. The average cell radius was determined to be R_{c}=7.5±1.3 μm, which compares well with previously reported radius values for the MCF-7 cell line (7.5-15 μm). The average microbead particle radius was determined via SEM to be 0.525±0.050 μm, consistent with the manufacturer's specifications of 0.525±0.015 μm. Furthermore, the volumetric magnetic susceptibility was determined to be 1.10±0.19, comparable to the manufacturer's reported value of 1.5.

TABLE 1

| | Variable | Average | Error^{a} | Units |
|---|---|---|---|---|
| Gap Distance | Z | 150 | 20 | μm |
| Cell Radius | R_{c} | 7.5 | 1.3 | μm |
| Particle Radius | Rₚ | 0.525 | 0.070 | μm |
| Volumetric | χ | 1.10 | 0.19 | a.u. |
| Magnetic Susceptibility | | | | |
| Binding Density^{b} | φ | 794 | 280 | particle cell⁻¹ |

^{a}Standard deviation
^{b}Obtained from flow cytometry reading

Approximation of the particle binding density per cell, φ, was then investigated. Under the ideal circumstances of complete surface area coverage of the cell surface, the maximum binding density of magnetic microparticles to one cell is determined to be 816±161 particles per cell. Determination of the actual particle-binding character of the cell must consider biological characteristics such as the number of possible binding sites (i.e. receptors) available on the cell surface and clustering of these binding sites. To this end, the determination of the number of receptors available on the cells was conducted via flow cytometry analysis. A cell suspension was incubated with the fluorescently-labeled antibody against EpCAM, a known antigen found on carcinoma cells, and subsequently analyzed via flow cytometry to yield a receptor number of 251,250±51,382 (approximately ±20%) EpCAM binding sites per cell, comparable to previous reports of 222,100±13,700 EpCAM receptors per cell. A second suspension of cells (26.25×10⁴ total cells) was then incubated with magnetic particles functionalized with anti-EpCAM at a concentration of 1 mg mL⁻¹ for 30 minutes; the tagged cells were then removed via centrifugation. A concentration of 1 mg mL⁻¹ magnetic microbeads in EMEM was demonstrated from flow cytometry experimentation with various particle suspensions to provide the maximum number of particles binding onto the cells.

To ensure complete separation of the cells from the magnetic beads in suspension, centrifugation of the entire suspension was performed. Centrifugation ensures that all cells in suspension (untagged and tagged) will be subsequently analyzed, while separating free magnetic microparticles from the bound cells. The cells recovered following centrifugation were incubated with anti-EpCAM-FITC, which will bind to any free, unoccupied receptors remaining on the cell. These suspended cells were then analyzed for the number of available receptors free of particles. After incubation the number of unoccupied EpCAM receptors was 6,898±1,218 EpCAM antigens per cell, which equates to an approximate 97% antibody coverage and an overall binding density of 794±280 microparticles per cell. To provide an independent confirmation of this value, the unbound microparticle suspension remaining after the centrifugation step was dried and weighed. A mass analysis was carried out, where the initial mass of particles that was incubated with 26.25×10⁴ cells was 1.1±0.1 mg and the remaining mass of cells after was determined to be 0.9±0.1 mg. As determined by hemacytometry, the approximate microparticle concentration of particles per mL of liquid stock is 8.45±1.33×10⁹ particles mL⁻¹, which translates to 8.45±1.33×10⁸ particles per mg of dry magnetic microparticles. Therefore, by mass conservation, a particle binding density of 644±338 beads per cell was determined, which is nearly 20% smaller than the coverage determined by flow cytometry but may be accounted for by dilution errors, hemacytometer errors, and the inaccuracies of the balance. However, comparison of the results attained from flow cytometry and mass conservation calculations illustrates that, within the margin of error, these two techniques yield consistent binding densities. This information is used to provide realistic constraints to the design of the microfluidic isolation device. Furthermore, understanding of the distribution in cell radius, magnetic microbead particle radius, and binding densities ensures that the resulting device is sufficiently robust enough to isolate all the cells of interest.

### Microfluidic Device Design Optimization

Employing the expression derived previously for the displacement of a cell-magnetic particle complex under the influence of a magnetic field and utilizing the values derived in Section V, Table 1, for the key parameters in the expression, it is now possible to computationally investigate the physical behavior and appropriate dimensions of the Generation I and Generation II cell separation device designs. Solving Eq. [20] for the length l_{ch} of the Generation I device with a reasonable lateral cell-particle complex displacement (Δx) of 100 μm and employing the commercial microbeads described earlier indicates that the applied current (I) would need to be greater than 10 A to produce a field sufficient to ensure that the length of the channel l_{ch} remained below one meter. Not only is this applied current value well above the constraints set by minimizing the Joule heating contribution, the derived device length of one meter is unrealistic. This result motivated the Generation II design, as derived in Eq. [24], which was designed to (i.) reduce the device length required for cell displacement and isolation relative to the Generation I design and (ii.) increase the magnetic forces experienced by the tagged target cells by virtue of its double current-carrying wire design that produces double the magnetic field. An average maximum cell displacement for the Generation II device, using the earlier-described parameters, was determined from Eq. [24]; as before, the intended design was envisioned to consist of a disposable microfluidic component and a re-usable electromagnetic component, with the length of the device set by the length of commercially-available glass slides (60 mm). To compute the lateral displacement of the cell-particle complex in the device, two current-carrying wires of equal dimension are placed at the outer edges of the device, the height of the channel (h) is assumed to be 50 μm, and the distance between the channel midpoint and the current-carrying wire array is set equal to the thickness of a #1 glass slide (z=150 μm). This lateral displacement was determined as a function of current and volumetric flow rate using a rearrangement of Eq. [24]. Volumetric flow rates are directly impacted by changes in channel width or starting position of the cell-particle complex; therefore, derivation of the cell-particle complex displacement as a function of volumetric flow rate is non-linear, and these two variables must be solved for simultaneously in the calculation. It should be noted that the calculated complex displacement is defined as the distance from the outside edge of the channel, near the wire, to the long axis of the microfluidic channel (FIG. 2C); therefore the width of the channel is equivalent to twice the displacement, as shown in FIG. 2C.

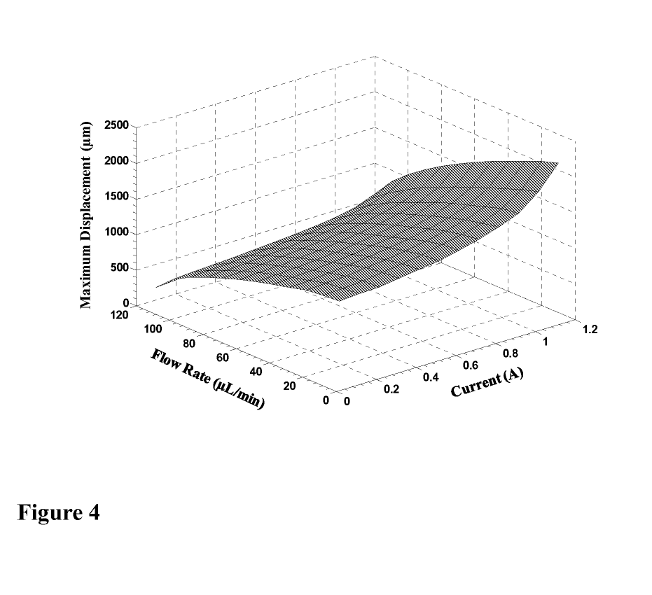

The surface plot shown in FIG. 4 illustrates the maximum displacement of an average cell-particle complex from channel edge to channel center as a function of current I and volumetric flow rate \dot{V} for the dual-wire device, as determined from Eq. [24]. All parameters fixed at average values in Table 1 and length of device constrained to 50 mm. Current varied from 0.1-1.2 A and volumetric flow rate varied from 10-1200 μL min⁻¹. Maximum displacement increases with increasing current and decreases with increases in flow rate. The displacement maxima, defined as the largest distances that the cell-particle complex traverses to reach the device center within a length of 50 mm, are below 2100 μm (or 2.1 mm), a width significantly less than that of a standard coverglass slide (24 mm). The device length was set to 50 mm to account for the integration of the channel outlet and inlets to create a hydrodynamic focusing of the buffer stream, as illustrated in FIG. 2A. Higher current through the device provides greater maximum cell displacement, as the magnetic force increases as I². The linear velocity of the cell in the \hat{x}-direction increases with increasing volumetric flow rates, causing a less drastic displacement as the particle travels down the microfluidic channel (ŷ-direction).

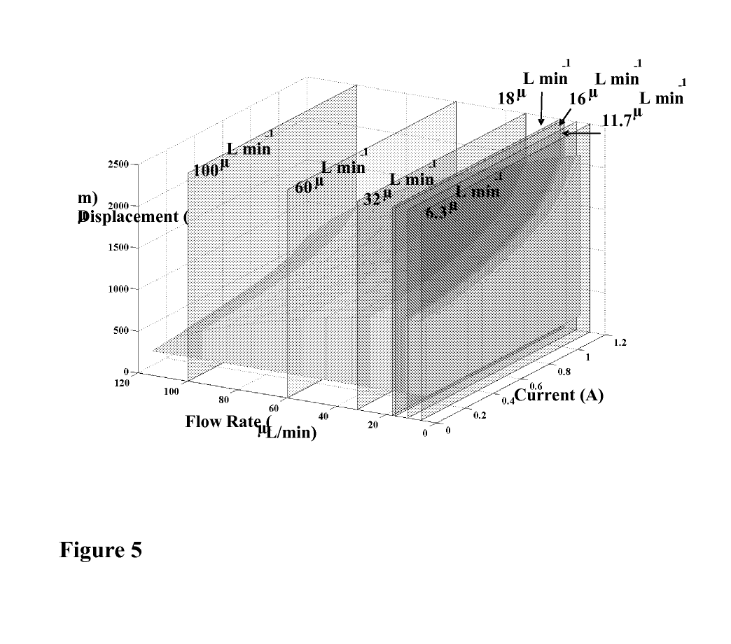

FIG. 5 depicts the surface plot from FIG. 4 along with intersecting planes that represent sample volumetric flow rates utilized with magnet-activated and non-magnetic cell separation systems described in the literature. FIG. 5 shows intersecting planes drawn at average throughputs for commercial magnet-based separation (600 μL min-1) and microfluidic cell separation devices (11.7-100 μL min-1). The 3-dimensional plot illustrates that rational design yields comparable throughputs, and narrow channel widths allow for greater throughputs than state-of-the-art separators. Other researchers who have isolated cells using commercial or microfluidic systems have employed volumetric flow rates on the order of 6.3 μL min⁻¹ to 100 μL min⁻¹. FIG. 5 illustrates that this design can effectively meet and exceed the processing speeds or throughputs of both commercial systems (600 μL min-1) and microfluidic devices (11.7-100 μL min-1). This is a relevant comparison because any new separation device must have at least the same throughput as similar, state-of-the art systems. Furthermore, control of the applied current and channel widths in this device allow for cell throughputs higher than those currently reported in the literature for other comparable devices.

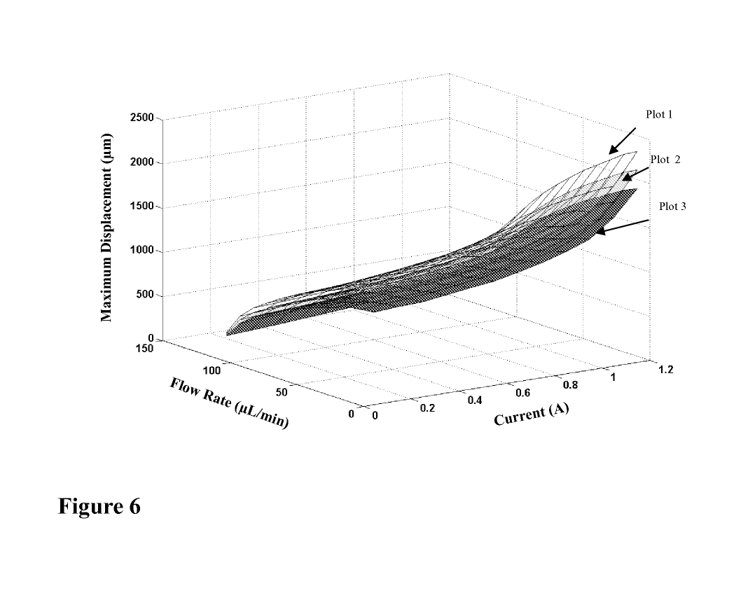

So far, all presented calculations were derived from average particle and cell characteristics shown in Table 1, whereas distributions in the values of these particular parameters were not considered. To address these parameter variations, the cell and particle diameter distributions shown in Table 1 were factored into the computational device design. This analysis demonstrates a need to design the dual-wire system according to the lower bound of the maximum cell-particle complex displacement as illustrated in FIG. 6, rather than target the average displacement of the complex as described earlier (FIGS. 4 and 5). To assess the worst-case scenario, or the lowest maximum displacement, the least favorable bounds of each of the parameters were inserted into the design equation (i.e. Rₚ=0.455 μm, R_{c}=8.8 μm, φ=514 particles cell⁻¹, χ=0.91). As it is desired that all cells be displaced from the sample stream into the collection stream located in the center of two sheath fluids (i.e. 100% recovery), the lower bound surface models the movement of a large cell with minimal magnetic-particle binding densities and thus minimal magnetic force experienced. Thus, the lowest surface plot shown in red in FIG. 6 represents the most conservative rational design criterion that should be followed for subsequent design of a magnetic-based cell separation platform. In FIG. 6, the average maximum displacement in FIG. 4 is re-plotted, shown in Plot 2. The upper bound plot (Plot 1) represents the case of highly mobile cell-particle complex, i.e. small cells maximally labeled with magnetic particles or best-case scenario (Rₚ=0.595 μm, R_{c}=6.2 μm, φ=1074 particles cell⁻¹, χ=1.29). The case where larger-than-average cells experience high drag, and are tagged with small particles with low susceptibility with minimum binding density (i.e. worst-case scenario), shown as Plot 3, represents the true realistic parameter space (Rₚ=0.455 μm, R_{c}=8.8 μm, φ=514 particles cell⁻¹, χ=0.91).

The parametric analysis conducted in this study illustrates that particle binding characteristics, as well as the individual cell and particle properties, do play an integral role in the separation efficiency. Unfortunately, most commercial MACS systems have been generalized for separation of a wide variety of cell population and are not tuned to account for these variations in particle-cell binding character. Therefore, this standardization of the commercial system may result in the low yields currently found for magnet-based cell isolations. Moreover, the total number of particles bound to a particular cell is a direct function of the cell type, the particle type, and the specific marker of interest. Therefore, prior to any experimentation, the number of particles bound to the cell should be directly measured, as presented in Section VI. A. and subsequently included in the validation studies presented below.

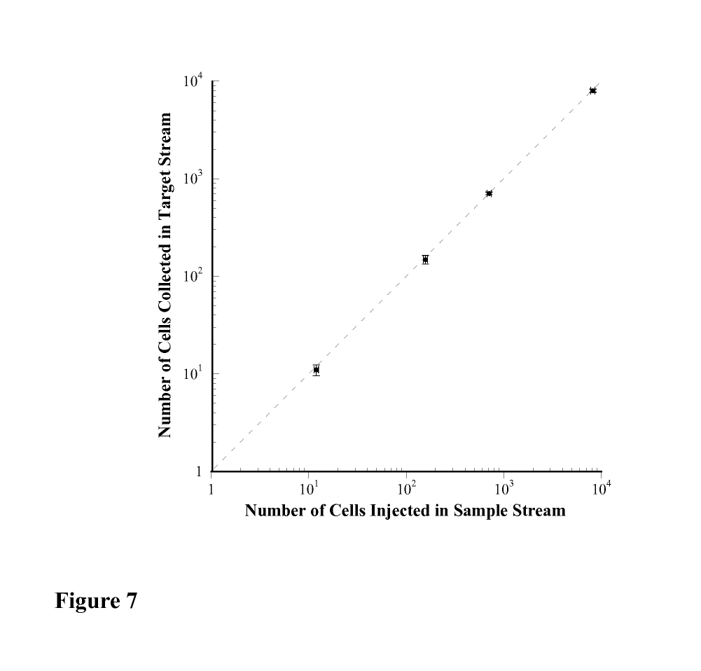

Following determination of the rational design criteria, preliminary validation studies of the sheath device were conducted with a homogeneous suspension of MCF-7 carcinoma cells in phosphate-buffered saline at a cell concentration of 1×10⁵ cell mL⁻¹. Several combinations of applied current, flow rate, and channel widths were investigated. By selecting specific combinations of these three variables, it is possible to probe designs that should separate all cells (below Plot 3 in FIG. 6), designs that should not separate the cells (above Plot 1 in FIG. 6), and design which should separate some of the cells (between Plot 1 and Plot 3 in FIG. 6). As shown in Table 2, the combinations predicted to separate all of the cells had efficiencies of approximately 100% within the margin of error, whereas combinations which forced the design outside of the optimized region yielded very low separation of labeled cells from the buffer solution. Interestingly, those parameters which intersect between the upper and lower bounds on FIG. 6 illustrate that only a percentage of the cells were actually separated from the fluid stream. Furthermore, as the parameter intersection approached the lower bound (or worst case-scenario) the percentage of cells increases towards 100% efficiency separation. This result illustrates that the surface plots shown in FIG. 6 accurately represent the available device design space, and given one or two parameters an optimized functional device can be attained. A second validation of the rational design was then probed as a mean to test cell concentration influences on the efficiency of separation for a single set of device parameters. A device made with optimal design parameters, as determined from the model and experimental preliminary validation (w=250 μm, \dot{V}=120 μL, min⁻¹ and I=0.25 A), was employed to test the efficiency of cell isolation as a function of MCF-7 concentration in buffer (10-10,000 cell mL⁻¹). As shown in FIG. 7, in this instance the efficiency of cell separation remains around 100% as the total number of cells injected is lowered from 10,000 cells to as low as 10 cells. As determined from the experiments shown in Table 2, all experiments were conducted with a 250 μm wide microfluidic channel at a flow rate 120 μL min⁻¹ and a current of 0.25 A. The four solid points represent five replicates experiments (n=5) for each respective cell suspension and the dotted line represents 100% efficiency of cell capture. Error bars represent the standard error of both the input cell number and collected cell number (n=5). Overall, the results of these validation tests illustrate that the device design optimized according to the derived computational model can effectively isolate (˜100%) of a magnetic-particle-tagged population of cells from a general cell suspension, even in low abundance.

### Example 2

### Microfluidic Device Design for Isolation of Rare Cell Populations

Example 2 describes the use of the rationally designed magnetophoretic microfluidic device described in Example 1 in the isolation of rare cell populations. First, the capabilities of the magnet-based separation device to extract single cancer cells from suspension, as well as high purity isolations of spiked cancer cells directly from whole blood, are described. In addition, the separation platform was used towards isolation of hematopoietic stem cells and endothelial progenitor cells from whole blood.

### Experimental Methods

Microfluidic Device Design and Fabrication.

To validate the developed optimized device design, microfluidic channels were fabricated as previously described. Plouffe et al. Langmuir 2007, 23, 5050; Xia et al. Angew. Chem. Int. Edit. 1998, 37, 551.

Wire arrays were designed using PCB123® printed-circuit board design software and ordered from Sunstone Circuits (Mulino, Oreg.). The wire dimensions were set to provide a gap encompassing the width of the device microfluidic channel; the height and width of the all of the wires were set to about 35 μm and about 178 μm, respectively. Teflon-insulated 18G copper wires were soldered to the ends of each of the printed circuit board arrays and the arrays were connected to a DC power supply (Elenco Electronics XP-4, Wheeling Ill.) that provided three fixed-current settings of 0.25 A, 0.50 A and 1.00 A via standard alligator clip connectors. The PDMS channels and wire arrays were visually aligned followed by injection of a prepared homogenous MCF-7 cell suspension using a syringe pump (Harvard Apparatus, Holliston, Mass.).

Microparticle Modification.

DynaBeads® MyOne™ Carboxylic Acid particles (Invitrogen, Carlsbad, Calif.) were modified with antibodies, either antibodies against the epithelial cell adhesion molecule (mouse anti-human EpCAM; Santa Cruz Biotechnology, Santa Cruz, Calif.) or antibodies against CD133 (mouse anti-human CD133, Miltenyi Biotec Inc, Auburn, Calif.) using standard carbodiimide chemistry (Hermanson, Bioconjugate Techniques; Academic Press: Boston, 1996) in ratios suggested by the reagent manufacturer (1:1 molar ratio of beads to protein; Pierce Biotechnology, Rockford, Ill.).

Spiked Cell Experiments in Buffer.

MCF-7 human breast adenocarcinoma cells (ATCC, Manassas, Va.) and human mature B-lymphoblast (Raji; ATCC) were cultured in 75-cm² tissue culture flasks at 37° C., 5% CO₂. MCF-7 cells were incubated in Eagle's Minimum Essential Medium (EMEM; ATCC) supplemented with 10% fetal bovine serum, 100 U mL⁻¹ penicillin, 100 μg mL⁻¹ streptomycin and 0.01 mg mL⁻¹ bovine insulin. Raji cells were incubated in RPMI-1640 (Mediatech, Herndon, Va.) supplemented with 10% fetal bovine serum, 100 U mL⁻¹ penicillin, and 100 μg mL⁻¹ streptomycin. Cells were grown to pre-confluence and isolated for experiments by trypsinization using a 0.25% Trypsin-EDTA solution. For preliminary microfluidic isolation validation experiments, cell suspensions were centrifuged at 190×g for 5 min, the supernatant was aspirated and then resuspended in 1× phosphate buffered saline (PBS) to remove dead cells and cell debris. The cells were resuspended at a concentration of approximately 10⁶ cells mL⁻¹ (measured using a hemacytometer).

Several different total numbers of MCF-7 cells (1000, 100, and 10 cells) were spiked into the Raji cell suspension prior to mixing with the Dynal® MyOne™ EpCAM-functionalized magnetic microbeads. The flow rates of the sheath fluid in the experiments were also varied within the constraints outlined in previous work by our group.³²

A Coulter counter/flow cytometer (Cell Lab Quanta™ SC; Beckman Coulter, Brea, Calif.) or a quantitative real-time reverse transcription-polymerase chain reaction (qRT-PCR) protocol (as described below) was used to count the number of target (MCF-7) cells that were separated from non-target (Raji) cells. A protocol, based on the distinct size difference of these two cells, was created to identify each cell population. The cells were gated by their electronic volume and granularity, and the total number of cells within the recovered suspension was assessed.

Enumeration of Purified MCF-7 Cells by RNA Isolation and Quantification.

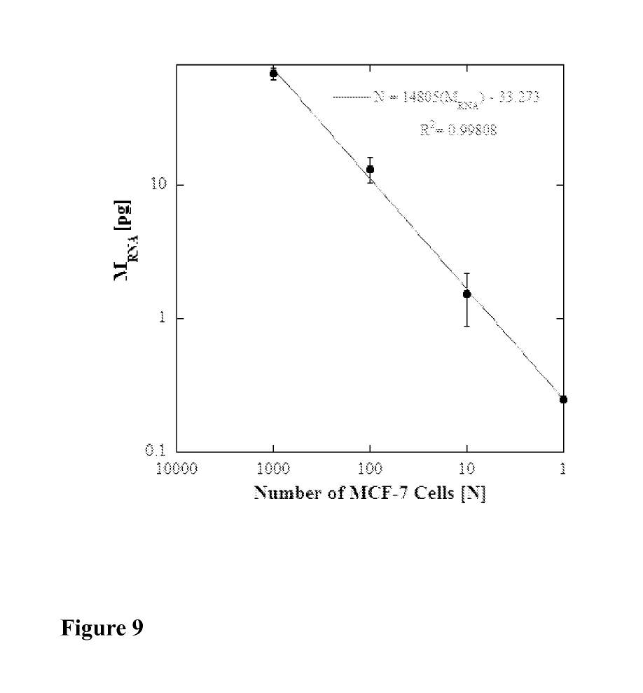

Cell dilutions from 10³ to 10⁰ cells mL⁻¹ were prepared by serial 10-fold dilution of suspensions with a concentration of 10⁴ cells mL⁻¹ in PBS in a total volume of 1 mL. Total RNA was isolated from the cell pellets using a method designed for rapid RNA isolation from low numbers of cells, the Absolutely RNA Nanoprep kit (Agilent, La Jolla, Calif.). The isolated RNA was detected by qRT-PCR using an assay to detect β(2)-microglobulin (β2m) housekeeping mRNA (assay ID Hs99999907_m1, Applied Biosystems, Foster City, Calif.). The mass of RNA isolated (ng) was determined at each MCF-7 cell density in triplicate. These values were used to generate a standard curve (FIG. 9) relating the total number of cells to a given RNA mass (pg). The RNA mass was used to determine the approximate number of cells retrieved from a device.

Viability of Recovered Cells.

Isolated cells were incubated with a 4 μM solution of EthD-1 (dead cell indicator) in PBS and a 2 μM solution of calcein (live cell indicator). Live and dead cells were counted using flow cytometry. To verify the health of the recovered cells, the cells from the target stream were centrifuged (along with any unbound particles displaced) and pipetted into a 96-well plate. The triplicate wells were then inspected and imaged 24 hr later to assess if the cells were healthy.

Spiked Cell Experiments in Blood.

As a further improvement on the heterogeneous suspension experiments described above and to more closely mimic the clinical setting, MCF-7 cells were spiked directly into whole blood. Whole blood was drawn from healthy volunteers and collected in EDTA-coated Vacutainer® tubes (Becton Dickinson, Franklin Lakes, USA). Approval from the Northeastern University Institutional Review Board was obtained for this purpose. No pre-processing, including erythrolysis, centrifugation, or dilution, was conducted on the obtained blood prior to spiking the MCF-7 cells and the EpCAM antibody-coated magnetic beads.

Prior to experiments, the location of the interface that forms between the injected blood and buffer was visually evaluated. As blood is a non-Newtonian, shear thinning fluid, it behaves differently from cells in buffer solutions and thus the required displacement for effective isolation is changed. The results of this evaluation influence the rational design optimization described previously. (Plouffe et al. Biomicrofluidics 2011, 5, 013413).

Whole Blood Cancer Cell Isolation.

As an experimental test to determine the capabilities of the fabricated microfluidic channel for rare-cell isolation, a concentration of 50 MCF-7 cells per mL was spiked into blood, followed by mixing in the Dynal® MyOne™ EpCAM functionalized magnetic microbeads. Unbound beads were allowed to remain in suspension during separation.

Isolation of Hematopoietic Stem Cells and Endothelial Progenitor Cells from Whole Blood.

To illustrate the utility of the magnetophoretic rational design in cardiovascular disease, we extracted hematopoietic stem cell (HSCs) and endothelial progenitor cells (EPCs) from whole blood using anti-CD133 functionalized microparticles. Again, whole blood was drawn from healthy volunteers and collected in EDTA-coated Vacutainer® tubes (Becton Dickinson, Franklin Lakes, USA).

Isolated cells were then labeled with additional antibodies to identify HSC and EPC populations. The HSCs were identified as labeling positive for mouse anti-human CD34 conjugated to fluorescein isothiocyanate (anti-CD34-FITC; Santa Cruz) and mouse anti-human CD45 conjugated to phycoerythrin (anti-CD45-PE; Santa Cruz), and negative for goat anti-human KDR (kinase insert domain receptor; Santa Cruz). The KDR was then conjugated to a secondary antibody donkey anti-goat peridinin chlorophyll protein (PerCP; R&D Systems, Minneapolis, Minn.). EPCs were identified as labeling positive for anti-CD34-FITC and anti-KDR-PerCP, and negative for anti-CD45-PE. Both cell populations were distinguished via a flow cytometer.

### Results and Discussion

As discussed in Example 1, rational magnetophoretic cell separation devices are disclosed that were designed using first-principles force balance calculations. As shown in FIG. 2, the computationally optimized device was comprised of two single wires with current applied in anti-parallel direction and a microfluidic straight channel chamber (FIG. 2A). The laminar flow nature of the micron-scale channel allows for the sheathing of a buffer stream with two sample streams. By selective labeling of cells in a heterogeneous cell suspension with superparamagnetic micron beads the magnetic field of the two wires allows for the selective displacement of the subsequently magnetic-tagged target cells from the two outside sample streams to a center buffer stream, as shown in FIG. 2B. The following Example illustrates that the optimized device effectively separated cells from suspension down to the single cell level. Furthermore, cancer cells were isolated from heterogeneous suspensions and whole blood in a high purity fashion. Finally, hematopoietic stem cells and endothelial progenitor cells were isolated from whole blood to illustrate the versatility of the device as a robust diagnostic platform and therapeutic monitoring tool.

In this Example, a magnet-based microfluidic device design was developed with optimized dimensions and operating conditions determined from a force balance equation that considers the two dominant and opposing driving forces exerted on a magnetic particle-tagged cell, the magnetic and viscous drag. The final microfluidic design was constrained to fit on a standard, commercially available, rectangular glass coverslip (60 (L)×24 (W)×0.15 (H) mm³) to accommodate small sample volume and point-of-care design considerations. Furthermore, as a means of minimizing bio-hazardous waste, in some embodiments, the microfluidic chamber was designed to be independent of the electromagnet (FIG. 2D). The anticipated performance of the device was examined via a parametric analysis of device width (w), applied current (I) and volumetric flow rate (\dot{V}).

Using the design described in Example 1, a device design with parameters of w=250 μm, \dot{V}=120 μL min⁻¹ and I=0.25 A was employed to test the efficiency of cell isolation as a function of MCF-7 concentration in buffer (1-1000 cell mL⁻¹). It should be noted that these parameters were chosen both via the computational design and due to the specific imposed constraints of the system, including glass coverslip dimensions and Joule heating. Higher flow rates (>120 μL min⁻¹) were computationally shown to require longer glass coverslips (>60 mm). Although larger glass substrates are contemplated, in one embodiment, the design is of a device using standard size components with a reasonable size to merit application in the clinic. Furthermore, by running the currents anti-parallel the volumetric flow rate can be maximized by having two sample streams and one central buffer stream. It can be shown by augmenting the design equation (Equation S4) to account for two currents running in a parallel fashion would allow for a central sample stream to displace to two outside collection streams with a greater magnetic force vector and a shorter device requirement. The Equation S4 is as follows:

$${F}_{m , x} = \frac{{V}_{p} {𝒳}_{p}}{{μ}_{o}} [ ( {B}_{x} ( x - X , z ) - {B}_{x} ( x + X , z ) ) ( \frac{\partial }{\partial x} \{ {B}_{x} ( x - X , z ) - {B}_{x} ( x + X , z ) \} ) + ( {B}_{z} ( x - X , z ) - {B}_{z} ( x + X , z ) ) ( \frac{\partial }{\partial z} \{ {B}_{x} ( x - X , z ) - {B}_{x} ( x + X , z ) \} ) ]$$

A side-by-side comparison still illustrates that within the required constraints a greater volumetric flow rate is more advantageous than a shorter glass coverslip requirement.

Inlet and outlet cell numbers were counted via two different techniques (i) using a Coulter counter and (ii) qRT-PCR. As shown in Table 2, the efficiency of isolation was above 95% for nearly all cell inlet concentrations regardless of enumeration platform. In addition, Table 2 illustrates that the magnetophoretic device is capable of single cell isolation from suspension (using the more precise qRT-PCR technique). To accurately count single cells from the collection stream using qRT-PCR, a calibration curve of mass of RNA (pg) versus cell number was first produced for 10³-10⁰ total cells, as described in the experimental methods section and illustrated in FIG. 9. The calibration curve then allows for the translation of extracted RNA mass to cell number for both inlet and outlet streams. These numbers were then compared with the Coulter counter numbers. It is clearly evident from Table 2 that both the Coulter counter and qRT-PCR can accurately identify cell numbers down to 10 cell mL⁻¹, but the Coulter counter fails at lower (single cell levels) whereas the qRT-PCR can ultimately count individual cells. Although qRT-PCR allowed for reliable cell counts less than 10 cells mL⁻¹, the Coulter counter yielded comparable counts ≧10 cell mL⁻¹ and thus was used for all subsequent experiments due to its rapid enumeration capabilities and ease of use versus qRT-PCR.

In Table 2, the number of MCF-7 cells injected and collected from the target stream outlet was counted via traditional Coulter counter enumeration. This number was compared with RT-PCR cell counts. The data represents triplicate experiments at inject cell concentrations of 1000, 100, 10, and 1 MCF-7 cell.

| Approx. No. | Inlet | | Outlet | | Efficiency | |
|---|---|---|---|---|---|---|
| Cells Injected | Coulter | qRT-PCR | Coulter | qRT-PCR | Coulter | qRT-PCR |
| 1000 | 1198 ± 28 | 1106 ± 168 | 1081 ± 38 | 1038 ± 7 | 90.2 ± 3.8 | 93.9 ± 14.3 |
| 100 | 137 ± 9 | 136 ± 11 | 135 ± 8 | 134 ± 5 | 98.5 ± 7.7 | 98.6 ± 7.7 |
| 10 | 20 ± 1 | 19 ± 3 | 19 ± 1 | 19 ± 0 | 95.0 ± 5.6 | 100.0 ± 15.8 |
| 1 | 0 ± 1 | 2 ± 0 | 0 ± 0 | 1 ± 0 | — | 50.0 ± 0.0 |

Coulter, Coulter Counter;
qRT-PCR, quantitative reverse transcription-polymerase chain reaction

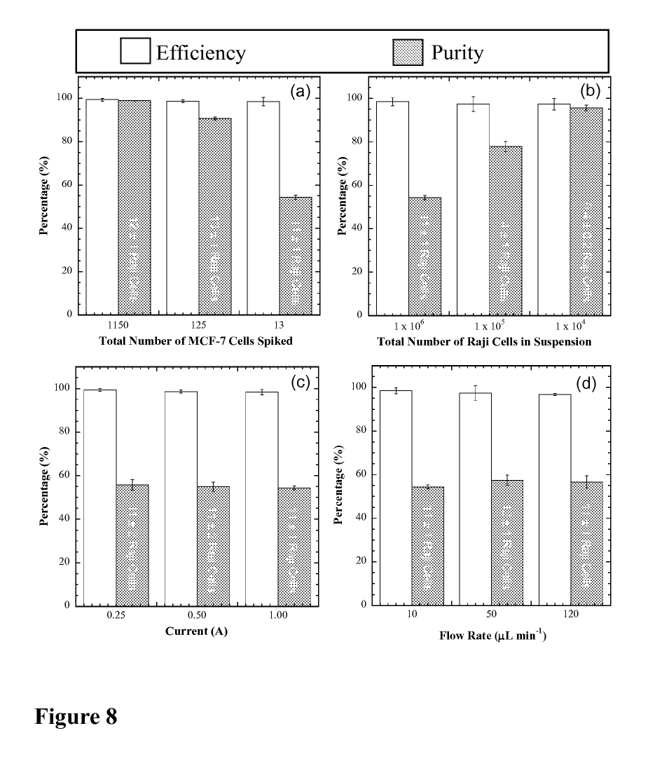

To investigate the influence of non-target cell populations on the overall efficiency, as well as to determine the estimated purity of the effluent stream, various heterogeneous cell suspensions were prepared. The first variable that was tested was the influence of target cell suspension (MCF-7 cancer cells) in a set non-target cell suspension (Raji B-lymphocytes) of 1×10⁶ cells per mL. The cancer cells spiked into a high concentration of B-cells represent a valid heterogeneous model for metastasis found in red cell-depleted blood. This experiment allows for the assessment of (i) the possibility of non-target cell labeling which would manifest itself in separation of the non-target cells along with target cells (or low purity yields) and (ii) the influence of non-target/target cell elastic collisions causing a reduction in efficiency or mechanical shifting of the non-target cells into the collection stream. As shown in FIG. 8A, the efficiency of isolation (shown in white) was above 95% for all conditions. On the other hand, it was observed that the purity of capture (defined as the percentage of non-target cells in the collected target stream) increased with increases in the total number of MCF-7 cells spiked into the Raji cell suspension. It should be noted though that the number of Raji cells collected was conserved at approximately 12 cells (˜0.001%). To test if this decreased purity is a result of either the target or non-target cells in suspension, experiments with differing number of non-target cells, where the target cell number is held constant at approximately 10 cells, were conducted. As shown in FIG. 8B, when the number of target cells (MCF-7 cells) was held at approximately 13±1 cells total and the non-target cell (Raji cells) number is decreased the percent purity of the isolated suspension increases to near 100%. This increase confirmed that the purity is not dependent on the target cells that are spiked into suspension, within the range of cell numbers investigated, but rather a direct function of the number of non-target in the sample stream. These results showed nearly 100% efficiency in the separation of about 10 cells in 10⁶ non-target cells.

To test if changing the parameters (i.e. flow rate and applied current) may result in higher or lower efficiencies and/or purities two additional experiments were devised. In the first experiment, the concentration of Raji cells was fixed at about 10⁶ with about 10 MCF-7 cells spiked into suspension; the flow rate was fixed at 120 μL min⁻¹ for both sample streams (a 240 μL min⁻¹ injection flow rate bifurcated into two side streams) and the applied current was tuned from 1.0-0.25 A. As illustrated in FIG. 8C, the efficiency and purity was not significantly changed, even though the magnetic force applied to the tagged cells was much greater. Conversely, in the second set of experiments, the concentrations were again held constant at 10⁶ Raji and 10 MCF-7 cells, with a constant current of 0.25 A; the flow rate V of the sample streams was now changed from 120-10 μL min⁻¹. It was observed, as shown in FIG. 2(d), that the flow rate also had no effect on the isolation efficiency and purity.

Overall, the experiments with heterogeneous suspensions illustrate that the rationally designed magnetophoretic separation platform is capable of high efficiency isolation down to the level of about 10 target cells in a 1 mL sample. Furthermore, it was illustrated that the purity within this device is a function of the number of non-target (or interfering) cells in the suspension. These results confirmed that the low isolations or recoveries reported in the literature can be attributed more to the lack of a rational design and less to the elastic collisions occurring with the separation platform.

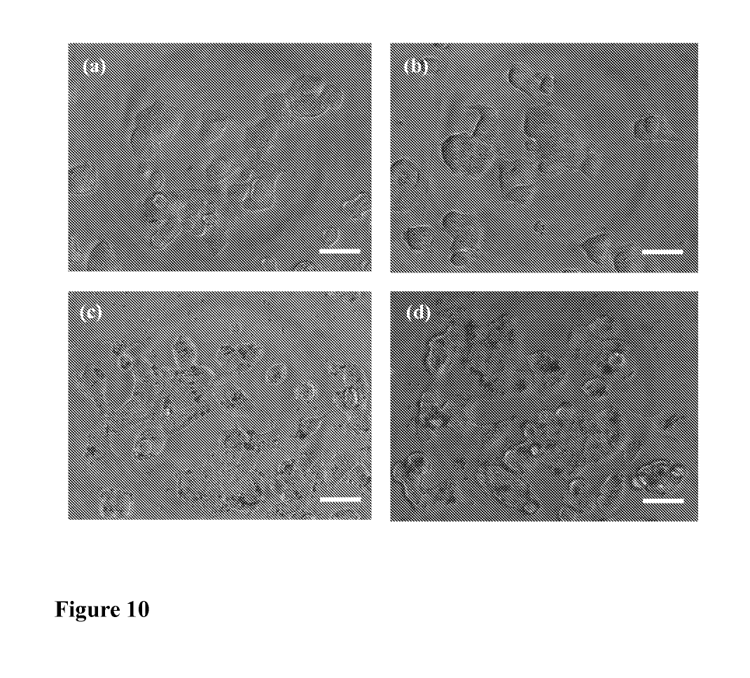

In addition to testing the influence of non-target cells on recovery and purity, a cell viability assay, using EthD-1 and calcein, was conducted on the outlet population and showed that the cells remained about 87.5±2.5% viable; this is within the range of the injected cell viability of 91.0±3.2%. This confirmed that the magnet-based cell separation platform presented does not adversely affect the cells. To further confirm that the particle tagging and/or high shear rates do not affect the growth and spreading of the cells, cells were plated for 24-hr in 96-well plates, followed by imaging and comparison with controls. No clear difference was observed in displaced cells versus non-displaced cells (see FIG. 10). Furthermore, the particle and the high flow rates have no visual influence on the behavior of the cells in culture. FIGS. 10A-D show the behavior of cells in culture to assess the impact of the separation process on the cells. FIG. 10A shows that a population of ˜1000 MCF-7 cells per 250 μL was plated as a comparative control. The control cells were judged against the cells depicted in FIG. 10C, which were incubated with particles but not run through the device. FIG. 10B depicts cells that were isolated using the device without particle attachment (no displacement). FIG. 10D depicts cells tagged with magnetic microbeads and displaced within the device. Scale bars represent 50 μm. Dark spots can be seen in FIGS. 10C-D, which indicate residual microparticles on the cell surface during culture and spreading.

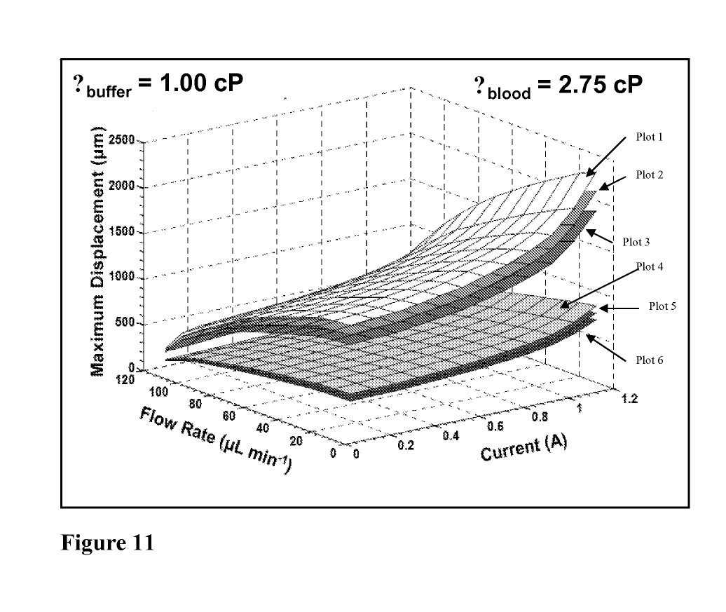

To further illustrate the capabilities of the described magnet-based microfluidic cell separation device, the model breast cancer MCF-7 cells were spiked into whole human blood at a concentration of 50 cells mL⁻¹. To determine the available device design space that computationally allows for efficient separation of the spiked cells from blood, parameters such as applied currents, volumetric flow rate and stream interfaces were evaluated. As described herein, blood behaves as a shear thinning fluid and within the context of microfluidic channel height range utilized in this study the red blood cell concentration (or hematocrit) causes a reduction in the apparent viscosity of the whole blood carrier fluid (Fahraeus-Lindqvist effect). Substituting a viscosity value for the η_{blood}=2.75 cP (as determined by calculations described herein) yielded a new surface plot as shown in FIG. 11, which illustrates the maximum displacement of an average cell-particle complex from edge to channel center as a function of applied current I and volumetric flow rate \dot{V} for the magnet-based microfluidic device described earlier.

In FIG. 11, the upper bound plot (Plot 4) represents the case of a highly mobile cell-particle complex, i.e. best-case scenario and the case where larger-than-average cells experience high drag and are tagged with small particles with low susceptibility with minimum binding density (i.e. worst-case scenario) is shown in Plot 6. The blood-based device, where η_{blood}=2.75 cP, was compared to a buffer-based device (Plots 1-3), with η_{buffer}=1.00 cP, illustrating a clear delineation of the blood from a solely buffer-buffer device. In a similar fashion, on the blood-based device, Plot 1 represents the best-case scenario, Plot 3 represents the worst-case scenario, and Plot 2 illustrates the mean displacement for an entirely buffer-based displacement scheme.

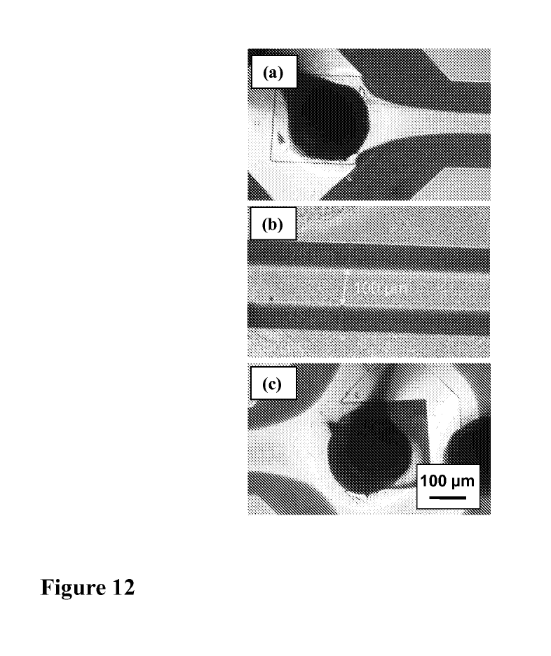

Subsequent to confining the flow rate and current, a second constraint, which must be determined experimentally, was applied to further refine the possible design space required for high purity separation. To maintain a continuous parabolic flow profile transversely across the channel, the location of the blood and buffer interface within the stream, sheath fluid flow pattern must be determined. To ensure complete separation of the target cells from the blood stream at the outer parameter of the device to the center collection buffer stream, the required displacement x must be well characterized and repeatable. Furthermore, to maintain the purity of the collection stream it is imperative that the blood streams not flow into the collection outlet. Tuning the buffer flow rate (from 120 μL min⁻¹ to 200 μL min⁻¹) to compensate for the higher viscosity of the blood allows the three streams to run parallel without mixing and for only the buffer collection stream to flow out of the center outlet (FIG. 12).

FIGS. 12A-C depict bright field micrographs illustrating the channels of the disclosed separation devices that include sample and buffer streams. FIG. 12A depicts the blood-buffer stream with a side channel flow of 120 μL min⁻¹ and a center buffer stream flow rate of 160 μL min⁻¹. FIG. 12B depicts the hydrodynamic focusing of the buffer stream between the two blood streams, where the buffer stream is approximately 100 microns in width, while the blood streams are both 75 microns in width. FIG. 12C depicts the blood streams being segregated from the collection outlet, which allows for only the target cells to be isolated. FIGS. 12A-C show that the flow rates used in the disclosed devices and methods result in a hydrodynamic focusing of the buffer between the two blood streams, such that the blood streams are separated from the collection outlet, allowing isolation of the target cells.

It was determined via visual inspection that buffer flow rates greater than 160 μL min⁻¹ were required to ensure (i) three separate side-by-side streams and (ii) no “leaking” of the blood stream into the collection outlet. Flow rates lower than 120 μL min⁻¹ resulted in a narrow buffer stream down the centerline, which ultimately increased the required displacement length and caused non-target cell contamination of the center target stream. It was also observed than flow rates greater 180 μL min⁻¹ resulted in a large buffer stream down the centerline, which dominated the microfluidic channel, potentially causing loss of target cells into the side, non-target outlets. Therefore for all further experiments, the buffer stream was injected at 160 μL min⁻¹ and blood was injected at 240 μL min⁻¹, resulting in two blood streams of 120 μL min⁻¹. Therefore for all further experiments, the buffer stream was injected at 160 μL min⁻¹ and blood was injected at 240 μL min⁻¹, resulting in two blood streams of 120 μL min⁻¹. The three stream widths were measured to determine the required displacement for labeled target cells to travel from the device edge to the long axis of the magnetophoretic device. The target cells would need to travel a total distance of 75 μm to enter the buffer stream for separation and isolation from the blood. Interestingly, when the three streams (at a buffer flow rate of 150 μL min⁻¹) had approximately equivalent widths of 83 μm, blood cells exited the center channel outlet. Therefore, the flow rate was increased to 160 μL min⁻¹ to ensure that pure populations were isolated. This phenomenon was not visualized in the heterogeneous validation experiments and thus was attributed to the high density of cells in blood, including nearly 5 billion red blood cells resulting in a non-Newtonian fluid sample.

Following augmentation of the rational design criteria for use with whole blood, validation studies of the sheath device were conducted. First the sheath characteristics of the device were validated via bright field imaging to determine the working range and ratios of flow rates. This was followed by a suspension of MCF-7 carcinoma cells spiked into whole human blood at a cell concentration of 50 cells mL⁻¹. Model systems of the kind employed here, specifically whole blood spiked with carcinoma cell lines, have been widely utilized in the optimization of diagnostic platforms. (Adams, A. A. et al, J. Am. Chem. Soc. 2008, 130, 8633; Du, Z. et al., Biosens. Bioelectron. 2006, 21, 1991; Mohamed, H. et al., J. Chromatogr. A 2009, 1216, 8289; Tan, S. J., et al., Biomed. Microdev. 2009, 11, 883; Wan, Y. A. et al., Cancer Res. 2010, 70, 9371.) Such models are reasonable given the wide variation in EpCAM antigen expression known to exist in CTCs in cancer patients. (Yu, M. et al., J. Cell Biol. 2011, 192, 373.). Specifically, while the current platform is optimized for MCF-7 cell isolation from whole blood, the device can easily be tuned to account for samples containing cells with lower EpCAM expression. Although EpCAM was chosen as the binding antigen—thus only isolating those cancers which express EpCAM—the magnet-based microfluidic platform was design in such a fashion that any cell to which a magnetic entity is bound can be separated and collected.

Three different applied currents were investigated. By selecting an applied current which fits within the determined design space it was shown that separation efficiencies above 95% could be achieved. As shown in Table 3, the applied currents (0.5 A and 1.0 A) that are predicted to separate all of the cells had efficiencies of approximately 90% within the margin of error. Whereas the current which predicts a lowest bound of 66.7 um from FIG. 11, less than the measured width of the blood stream of 75 um, yielded lower separation of labeled cells from the blood (˜87%). Interestingly, as the applied current was increased, the purity of capture inversely decreased. It is known that red blood cells are paramagnetic and white blood cells are diamagnetic in oxygenated blood. (Melville et al. IEEE Trans. Magn. 1975, 11, 1701). This may explain the increase in non-target cells in the collection stream. Higher currents produce higher magnetic fields, causing the white blood cells to remain in the blood stream and to even be attracted to the edges of the channel due to their diamagnetic behavior. Furthermore, in vitro experiments assessing the radial distribution of white blood cells in small glass tubes (69 um to 200 um diameter) have shown that white blood cells marginate in a tube depending on rheological factors such as hematocrit, blood suspension medium and shear stress. (Goldsmith et al., Microvas. Res. 1984, 27, 204.). White blood cell margination has also been shown in large rectangular channels (3 mm wide and 300 um deep), also dependent on blood rheology. (Abbitt et al., Am. J. Physiol. Heart Circ. Physiol. 2003, 285, H229; Jain and Munn, PLoS ONE 2009, 4, e7104). Conversely, in the presence of the applied magnetic field, the paramagnetic behavior of the red blood cells would result in a very small population displacing from the blood stream to the buffer stream.

Table 3 shows capture efficiency and purity with a spiked concentration of 50 MCF-7 cells mL⁻¹ in whole human blood. The lowest bound computationally optimized is also shown alongside the efficiency and purity.

| Current | Lowest Bound | Efficiency | Purity |
|---|---|---|---|
| (A) | (μm)* | (%) | (%) |
| 0.25 | 66.7 | 87.5 ± 3.2 | 81.0 ± 2.6 |
| 0.50 | 308.5 | 93.2 ± 4.5 | 78.4 ± 4.6 |
| 1.00 | 561.6 | 94.7 ± 3.4 | 55.6 ± 5.2 |

*From FIG. 11

To illustrate how small a population is shifting from the side stream to the center stream, blood is composed of 10⁹ red blood cells per mL and only 45-60 red blood cells are found in the collection stream after processing a 1 mL blood sample. Referring back to the heterogeneous suspensions shown in FIG. 8, it is apparent that the diamagnetic behavior of white blood cells did not seem to have an influence on the purity of the collected stream with changes in applied currents. Overall these results illustrate that lower currents sacrifice efficiency but are required to isolate a pure population. Depending on the desired end results, either high current should be used for high efficiency or low current for high purity.

In addition to isolation of cancer cells from heterogeneous suspensions in buffer and blood, the microfluidic magnet-based separation platform was utilized for the collection of CD133+ stem cells directly from whole blood. Two different cell populations in the blood are known to express CD133 antigen, endothelial progenitor cells (EPCs) and hematopoietic stem cells (HSCs).⁵⁵ EPCs represent a population of rare cells that circulate in the blood with the ability to differentiate into endothelial cells that make up the lining of blood vessels. Therefore endothelial repair dysfunction results in changes in circulating EPCs which correlate with cardiovascular risk and clinical outcome. Thus EPC number may serve as a valuable biomarker for cardiovascular risk assessment, disease progression and response to therapy (Hill et al., New Engl. J. Med. 2003, 348, 593; Van Craenenbroeck, et al., J Immunol Methods 2008, 332, 31; Werner et al., G. New Engl J Med 2005, 353, 999). On the other hand, HSCs are a circulating stem cell population that give rise to the all the cell types in the blood (i.e. red blood cells, white blood cells, and platelets). HSCs can be derived from whole blood, bone marrow, and umbilical cord blood. Isolation of HSCs directly from whole blood represents an attractive cell source that is readily available and can be collected noninvasively. This particular stem cell population has shown great promise in autologous transplantation for autoimmune disorders (Gratwohl et al., Bone Marrow Transpl. 2005, 35, 869) and treatment of blood-origin cancer. (Harousseau, et al., P. New Engl. J. Med. 2009, 360, 2645; Michallet, M. et al., Blood 2011, 117, 1516). Therefore, the instantly-disclosed devices and methods satisfy a great need exists to isolate the EPCs and HSCs from whole blood.

To achieve selective isolation of the two CD133+ cells, the superparamagnetic microbeads were functionalized with antibodies against CD 133 and mixed into whole unprocessed blood. After separation the cells were then immunofluorescently stained with antibodies against CD34, CD45, and kinase insert domain receptor (KDR, also called vascular growth factor receptor 2). The EPCs were defined as CD133+/CD34+/CD45−/KDR+ and the HSCs were defined as CD133+/CD34+/CD45+/KDR−.⁵⁵ As described above, the design criteria was tuned to account for a blood-buffer system, therefore buffer was run at a flow rate of 160 μL min⁻¹ and the blood was run at 120 μL min⁻¹. As shown in Table 4, the total number of HSCs and EPCs, as counted in a flow cytometer, in one milliliter of whole blood was shown to be 6753±251 and 1190±102 cells, respectively. Enumeration of the effluent stream illustrated a consistent number of HSCs was isolated at all investigated currents. On the other hand, the number of EPCs collected increased with increasing current. Table 4 shows that at all currents approximately 94-99% of the all the HSCs were isolated, where EPCs isolation efficiency increased from 59.8±6.5% at I=0.25 A to 95.5±3.1% at I=1.0 A. This difference is hypothesized to be a result of higher CD133 antigen densities on HSCs versus EPCs. A higher antigen density would result in larger overall bead density, increasing the magnetic force even at lower currents. Conversely, the hypothesized low density of CD133 on the EPCs results in a low magnet force on the cells at low currents, but at a sufficiently high enough magnetic force at higher currents.

Table 4 shows hematopoietic stem cells and endothelial progenitor cells were isolated from whole human blood using CD 133+ functionalized magnetic microparticles. The data represents five replicates at each applied current.

TABLE 4

| | | Cells Counted | Efficiency (%) |
|---|---|---|---|
| Inlet^{†} | Total | — | — |
| | HSC* | 6753 ± 251 | — |
| | EPC^{#} | 1190 ± 102 | — |
| I = 0.25 A | Total | 14496 ± 409 | — |
| | HSC* | 6499 ± 192 | 96.2 ± 2.9 |
| | EPC^{#} | 711 ± 77 | 59.8 ± 6.5 |
| I = 0.50 A | Total | 15026 ± 473 | — |
| | HSC* | 6507 ± 237 | 96.4 ± 3.5 |
| | EPC^{#} | 978 ± 64 | 82.3 ± 5.3 |
| I = 1.00 A | Total | 15349 ± 369 | — |
| | HSC* | 6514 ± 155 | 96.5 ± 2.3 |
| | EPC^{#} | 1137 ± 37 | 95.5 ± 3.1 |

HSC, hematopoietic stem cells;
EPC, endothelial progenitor cells
^{†}Measured using flow cytometry
*CD133+/CD34+/KDR−/CD45+
^{#}CD133+/CD34+/KDR+/CD45−

### Discussion

Example 1 shows that the disclosed devices and methods efficiently separates rare cells. A robust magnet-based microfluidic platform for malignancies and cardiovascular disease diagnostics and therapeutic monitoring was described. Using the rational design described in Example 1, single cells were shown to be efficiently isolated from suspension. Furthermore, high purity isolation of cancer cells from heterogeneous suspensions, both in buffer and whole blood, was achieved. Finally, EPCs and HSCs were isolated from whole human blood in a rapid and efficiency fashion. Overall, the instant device illustrates an efficient separation platform for high purity, efficient, and rapid collection of rare cells populations.

### Computational Rational Design Optimization Calculations

Detailed calculations of the computational rational design optimization, the calibration graph of the qRT-PCR data (FIG. 9), culture of the cells with and without magnetic bead labeling after separation (FIG. 10), average maximum displacement for a blood-based displacement platform (FIG. 11) and brightfield micrograph of the blood-buffer (side-by-side) flow in the microfluidic channel (FIG. 12) are described. FIG. 9 is a quantitative reverse transcription-polymerase chain reaction (qRT-PCR) standard curve relating total number of cells (N) to a corresponding mass of RNA (M_{RNA}) value. Total RNA was isolated from the cell pellets using a method designed for rapid RNA isolation from low numbers of cells, the Absolutely RNA Nanoprep kit. The isolated RNA was detected by qRT-PCR using an assay to detect β(2)-microglobulin housekeeping mRNA.

This section describes the formulation of magnetically-labeled cell displacement in a channel, subjected to both magnetic \vec{F}ₘ and Stokes \vec{F}ₛ forces, for utilization in the design of the current magnetophoretic cell separator. The magnetic force and the Stokes force contributions are considered separately; note that gravity and buoyancy forces are negligible and are thus not considered here. The trajectory of a magnetically-labeled cell in the proposed microfluidic device is modeled by evaluating the forces on the cell generated by motion through the fluid under the attractive action of a magnetic field. The displacement is derived for a simply buffer-based, Newtonian system, followed by derivation of the viscosity of a blood-based system, a well known non-Newtonian fluid. To facilitate the discussion of the influence of non-Newtonian fluid dynamics on the overall microfluidic device a brief description of the force balance is provided below.

First, a single magnetic particle is idealized as a magnetic sphere of uniform moment density. The magnetic force exerted on the particle, \vec{F}ₘ=(\vec{m}·∇)\vec{B}, is evaluated from the overall moment on the particle \vec{m}=Vₚ\vec{M} where Vₚ is volume of the particle and the volume magnetization is \vec{M}. The value of \vec{M} is equal to Δχ\vec{H}, where Δχ is the volumetric magnetic susceptibility difference between the particle (χₚ) and the surrounding buffered fluid medium (χ_{med}), and \vec{H} is the applied magnetic field strength. The overall response of a magnetic particle in a fluid to a magnetic field is then determined by the strength and gradient of the applied magnetic field (\vec{B}=μₒ\vec{H}), yielding (Boyer, T. H. Am. J. Phys. 1988, 56, 688; Lee et al., Appl. Phys. Lett. 2004, 85, 1063) the following equation:

$${{F}^{->}}_{m} = \frac{{V}_{p} {Δ}_{𝒳}}{{μ}_{o}} ( {B}^{->} \cdot ∇ ) {B}^{->} \qquad [ 1 ]$$

where μₒ is the permeability of vacuum equal to 4π×10⁻⁷T m A⁻¹. The applied magnetic field can be solved using the Biot-Savart Law. (Tipler, P. A.; Mosca, G. Physics for Scientist and Engineers; 5th ed.; W.H. Freeman and Company: New York, 2004).

In opposition to the magnetic force \vec{F}ₘ acting on the magnetic particles, there exists a viscous drag force \vec{F}ₛ acting on the particle in the direction opposite to the particle motion. (Bird et al., Transport Phenomena; 2nd ed.; John Wiley & Sons, Inc.: New York, 2002). This Stokes force \vec{F}ₛ is dependent on the suspension medium viscosity (η), the particle radius (Rₚ), and the velocity \vec{v} of the particle in the direction of the magnetic force. The inertial effects on the particles suspended in the fluid are negligible, as the Reynold's number is less than unity due to the geometric constraints of the microfluidic chamber. This result implies that while the fluid exerts drag on each particle, the particles exert no force on the fluid. (Sinha, A, et al., J. Magn. Magn. Mater. 2009, 321, 2251; Sinha, A. et al. Phys. Fluids 2007, 19). Moreover, we assume a dilute particle suspension, thus no short-range inter-particle dipolar interaction exist. As the magnetic particle is carried by the flowing solution in the ŷ-direction, within the context of laminar flow, the hydrodynamic forces only act in the \hat{x}-direction (direction perpendicular to flow). This force can be expressed as:

\vec{F}ₛ=−6πηRₚv\hat{x} [2]

The overall force on the magnetic particle is the sum of the magnetic force \vec{F}ₘ and the hydrodynamic force \vec{F}ₛ that lends a constant velocity to the particle, which explicitly sets the acceleration equal to zero:

\vec{F}ₘ+\vec{F}ₛ=0 [3]

Thus to determine the resultant magnetic field generated by the two conducting strips shown in FIG. 2, an array of conductors is considered where one conductor is positioned at far edge of the microfluidic channel with current flowing in the negative ŷ-direction and the other conducting wire at the alternate edge of the microfluidic channel with current in the positive ŷ-direction, as shown in FIG. 2C. Solving the magnetic force equation for the two wire array yields the expression for the magnitude of the attractive force exerted on a magnetic particle in the double-wire sheath device design configuration:

$${F}_{m , x} = \frac{{V}_{p} {𝒳}_{p}}{{μ}_{o}} [ ( {B}_{x} ( x - X , z ) - {B}_{x} ( x + X , z ) ) ( \frac{\partial }{\partial x} \{ {B}_{x} ( x - X , z ) - {B}_{x} ( x + X , z ) \} ) + ( {B}_{z} ( x - X , z ) - {B}_{z} ( x + X , z ) ) ( \frac{\partial }{\partial z} \{ {B}_{x} ( x - X , z ) - {B}_{x} ( x + X , z ) \} ) ] \qquad [ 4 ]$$

Substituting this magnetic force equation into the force balance (Eq. [3]) allows for optimization of the applied currents and cell throughputs.

### Influence of Whole Blood on Rational Design

Secondly, to address the added complexity of using Non-Newtonian fluids, such as blood, the model framework was augmented. The primary parameter that impacts the overall implementation of the magnetophoretic device described above for a blood-based design is the viscosity of the carrier fluid (η) in which the target cells are located. This viscosity component plays a critical role in the drag force experienced by the cell during displacement and may impact the interface location (i.e. blood-buffer), causing a readjustment in the displacement parameter x, in Eq. [4], compared with the buffer-based displacement design.

To further complicate the determination of apparent blood viscosity (η_{blood}), there is a known microcirculatory phenomenon called the Fahraeus-Lindqvist effect that leads to a reduction in the proportion of blood volume occupied by red blood cells (or hematocrit, H_{c}) in small arterioles less than 200 microns in diameter and capillaries relative to the hematocrit of large feed arteries. This decrease in hematocrit in these flow vessels reduces the relative blood viscosity in the small vessels, which helps to offset the increase in viscosity that can occur because of reduced velocity in these same vessels. The net effect of these changes is that blood flow in the microcirculation has a lower viscosity than what is predicted by in vitro blood viscometer measurements.

Therefore, to best estimate the viscosity of blood in the present work a parametric description of apparent blood viscosity relative to the viscosity of plasma (ηₚₗₐₛₘₐ=1.5 cP) as a function of channel height (h_{ch}, in microns) and hematocrit, H_{c}, was chosen. This description was established by Pries et al. from a collection of 18 separate studies. (Pries, A. R. et al. Cardiovasc. Res. 1996, 32, 654). Assuming velocities higher than 50 channel heights per second, the apparent viscosity was empirically determined to be:

$$\begin{matrix}{η}_{blood} = {η}_{plasma} [ ( 1 + ( {η}_{0.45} - 1 ) \frac{{( 1 - {H}_{c} )}^{C} - 1}{{( 1 - 0.45 )}^{C} - 1} ) {( \frac{{h}_{ch}}{{h}_{ch} - 1.1} )}^{2} ] \times {( \frac{{h}_{ch}}{{h}_{ch} - 1.1} )}^{2} \\ where \text{:} & [ 5 ] \\ {η}_{0.45} = 6 {e}^{- 0.085 {h}_{ch}} + 3.2 - 2.44 {e}^{- 0.06 {h}_{ch}^{0.645}} & [ 6 ] \\ C = ( 0.8 + {e}^{- 0.075 {h}_{ch}} ) ( \frac{1}{1 + {10}^{- 11} {h}_{ch}^{12}} - 1 ) + \frac{1}{1 + {10}^{- 11} {h}_{ch}^{12}} & [ 7 ]\end{matrix}$$

Here, the variable η_{0.45} describes the apparent viscosity of at a phenotypical hematocrit (H_{c}=0.45) as a function of height (in microns). This empirical expression is a necessary aspect to designing a realistic model of cell displacement from a blood medium to the buffer stream. The resulting η_{blood} can then be directly substituted into Eq. [3] in place of the general suspension medium viscosity term η. Overall the change in viscosity results in a linear change in the ultimate design criteria. Accounting for this augmentation in the design space via the viscosity parameter at the onset allows for a single fabrication of a device without extraneous experimentation and optimization of the flow rate and/or applied current parameters.

As the empirical model (Eqs. [5-7]) formulated by Pries et al.⁹ is predicated on the assumption that blood's linear velocities are higher than 50 channel heights per sec within the channel, a calculation of the flow velocities in the channel is required prior to computing the viscosity of whole blood. The device utilized throughout this report was ˜50 microns in height; thus for implementation of the empirical model, the linear velocity of the blood must be in excess of 2500 μm³ sec⁻¹. Calculations for a 1000 micron wide channel (the largest channel in the study) with a flow rate of 10 μL min⁻¹ (the lowest volumetric flow rate in this study) yield a fluid linear velocity of 10⁴ μm³ sec⁻¹ down each side channel. This flow rate (10⁴ μm³ sec⁻¹) is significantly higher than the fluidic velocity assumption defined in the empirical model (2500 μm³ sec⁻¹); thus the estimate for the apparent blood viscosity present in the microfluidic device is valid. Therefore, the constants C from Eq. [7] and η_{0.45} from Eq. [6] were determined with h_{ch}=50 microns to be 0.7765 and 2.13, respectively. Inserting these values into Eq. [5] at an average phenotypical hematocrit (H_{c}) of 0.45 yields an apparent blood viscosity η_{blood} of 2.75 cP.

Other aspects, modifications, and embodiments are within the scope of the following claims.

1. A magnetophoretic separation device comprising:
(a) a separation chamber comprising a plurality of channels that provide two or more streams, the streams comprising:
(i) a sample stream comprising target biological materials and non-target biological materials, wherein the target biological materials are magnetically-labeled; and
(ii) a buffer stream that is substantially free of the sample;
wherein the one or more streams combine in a single collection channel without fluidic mixing; and
(b) one or more wire(s) carrying a current, the wires generating a magnetic force that deflects the one or more magnetically-labeled target biological materials into the buffer stream.

2. The device of claim 1, wherein the one or more wire(s) carrying a current is a single wire.

3. The device of claim 1, wherein the one or more wire(s) carrying a current are two wires, wherein the first wire is in parallel alignment with the second wire.

4. The device of claim 1, wherein the one or more wire(s) carrying a current are more than two wires.

5. The device of claim 1, further comprising an alignment guide that aligns the separation chamber with the one or more wires.

6. The device of claim 1, wherein the separation chamber is separated from the one or more wires by a vertical distance of about 10 microns to about 500 microns.

7. The device of claim 6, wherein the target biological materials are cells, proteins, solutes, or particulates susceptible to a magnetic field.

8. The device of claim 6, wherein the target biological materials are from peripheral whole blood, tissue digestate, amniotic fluid, umbilical cord blood, fine needle aspirates, vitreous humor biopsies, cerebrospinal fluid, or other biological fluids.

9. The device of claim 7, wherein the cells are rare cells compared to the total number of cells in the sample.

10. The device of claim 9, wherein the rare cells are peripheral hematopoietic stem cells, endothelial progenitor cells, circulating tumor cells, mature circulating endothelial cells, amniotic stem cells, mesenchymal stem cells, adipose-derived stem cells, intestinal stem cells, skin stem cells, neural stem cells, cancer stem cells, adult stem cells, fetal stem cells, or progenitor cells.

11. A method of separating target biological materials from non-target biological materials in a sample, the method comprising:
(a) labeling the target biological materials in a sample with a magnetic tag;
(b) introducing the sample into at least a first inlet of at least a first channel in a magnetophoretic separation device;
(c) introducing a buffer into a second inlet of a second channel of the magnetophoretic separation device;
(d) generating a magnetic force by providing a current in one or more wire(s) placed adjacent to the channels, thereby deflecting the labeled target biological materials into the channel carrying the buffer; and
(e) collecting the target biological materials from an outlet of the second channel.

12. The method of claim 11, wherein the one or more wire(s) carrying a current is a single wire.

13. The method of claim 11, wherein the one or more wire(s) carrying a current are two wires, wherein the first wire is placed in parallel alignment with the second wire.

14. The method of claim 11, wherein the one or more wire(s) carrying a current are more than two wires.

15. The method of claim 11, wherein the target biological materials are from peripheral whole blood, tissue digestate, amniotic fluid, umbilical cord blood, fine needle aspirates, vitreous humor biopsies, cerebrospinal fluid, or other biological fluids.

16. The method of claim 11, wherein the target biological materials are cells, proteins, solutes, or particulates susceptible to a magnetic field.

17. The method of claim 16, wherein the cells are rare cells compared to the total number of cells in the sample.

18. The device of claim 17, wherein the rare cells are peripheral hematopoietic stem cells, endothelial progenitor cells, circulating tumor cells, mature circulating endothelial cells, amniotic stem cells, mesenchymal stem cells, adipose-derived stem cells, intestinal stem cells, skin stem cells, neural stem cells, cancer stem cells, adult stem cells, fetal stem cells, or progenitor cells.

19. A method of constructing a magnetophoretic separation device, comprising:
(a) providing a substrate;
(b) constructing a separation chamber on the substrate, wherein the separation chamber comprises a plurality of channels, wherein one or more sample channels combine with a buffer channel in a single collection channel; and
(c) constructing one or more wire(s) carrying a current on the substrate adjacent to the separation chamber.

20. The method of claim 19, wherein the one or more wire(s) carrying a current is a single wire.

21. The method of claim 19, wherein the one or more wire(s) carrying a current are two wires, wherein the first wire is in parallel alignment with the second wire.

22. The method of claim 19, wherein the one or more wire(s) carrying a current are more than two wires.

23. The method of claim 19, further comprising constructing an alignment guide that aligns the separation chamber with the one or more wires.

24. The method of claim 23, wherein the separation chamber is separated from the one or more wires by a vertical distance of about 10 microns to about 500 microns.
